(19) United States
(12) Patent Application Publication
(10) Pub. No.: US 2019/0343985 A1
(43) Pub. Date: Nov. 14, 2019

(54) A SYNTHETIC IMPLANTABLE SCAFFOLD

(71) Applicant: COMMERCIAL DEVELOPMENT & INDUSTRY PARTNERSHIPS, New South Wales (AU)

(72) Inventors: Hala ZREIQAT, Chatswood (AU); Seyed Iman ROOHANIESFAHANI, Ryde (AU); Yogambha RAMASWAMY, Kensington (AU); Young Jung NO, North Strathfield (AU)

(21) Appl. No.: 16/475,051

(22) PCT Filed: Dec. 28, 2017
(86) PCT No.: PCT/AU2017/051466

§ 371 Date: Jun. 28, 2019

(30) Foreign Application Priority Data
Dec. 30, 2016 (AU) 2016905392

(51) Int. Cl. A61L 27/16; A61L 27/22; A61L 27/52; A61L 27/10

(52) U.S. Cl. CPC A61L 27/16; A61L 27/222; A61L 27/306; A61L 27/10; A61L 27/52

(57) ABSTRACT

The present invention provides a synthetic implantable scaffold comprising a plurality of polymer fibres in contact with a composition comprising a hydrogel-forming polymer and a biocompatible ceramic material. Preferably the polymer fibres are formed from ultra-high molecular weight polyethylene (UHMWPE) and are in the form of a bundle of fibres. Preferably the implantable scaffold comprises a plurality of bundles of individual polymer fibres, which may be in the form of a braid. The hydrogel-forming polymer is preferably polyvinyl alcohol for mimicking the fibre-ECM hierarchical structure of native tendons or ligaments. The biocompatible ceramic material is preferably Hardystonite (Ca₂ZnSi₂O₇) doped with Sr, Mg or Ba. The synthetic implantable scaffold of the invention is particularly suited as a synthetic ligament or tendon. The invention also relates to a method for preparing a synthetic implantable scaffold, and use of the implantable scaffold for partial or full tendon or ligament repair.

This application claims priority to and the benefit of Australian provisional patent application no. 2016905392 dated 30 Dec. 2016, which is incorporated herein by cross-reference in its entirety.

### FIELD OF THE INVENTION

The present invention relates to tissue engineering, and in particular relates to a synthetic implantable scaffold that can be installed in vivo to repair or replace a ruptured or diseased tissue, such as a ligament or tendon. In particular, the invention relates to the provision of a synthetic tendon or ligament that is biocompatible and that reproduces closely the mechanical properties of native ligaments and tendons. However, it will be appreciated that the invention is not limited to this particular field of use.

### BACKGROUND OF THE INVENTION

The following discussion of the prior art is provided to place the invention in an appropriate technical context and enable the advantages of it to be more fully understood. It should be appreciated, however, that any discussion of the prior art throughout the specification should not be considered as an express or implied admission that such prior art is widely known or forms part of common general knowledge in the field.

A tendon is a tissue that attaches a muscle to other body parts, usually bone, and is the connective tissue that transmits the mechanical force of muscle contraction to the bone. The tendon is firmly connected to muscle fibres at one end and to components of the bone at its other end. Tendons are remarkably strong, having one of the highest tensile strengths found among soft tissues. Their great strength, which is necessary for withstanding the stresses generated by muscular contraction, is attributed to the hierarchical structure, parallel orientation, and tissue composition of tendon fibres.

A tendon is composed of dense fibrous connective tissue made up primarily of collagenous fibres. Primary collagen fibres, which consist of bunches of collagen fibrils, are the basic units of a tendon, and typically have a diameter of 5 to 30 micrometers. Primary fibres are bunched together into primary fibre bundles (subfasicles), groups of which form secondary fibre bundles (fasicles), and typically have a diameter of 150 to 1000 micrometers. Multiple secondary fibre bundles form tertiary fibre bundles, which typically have a diameter of 1000 to 3000 micrometers, groups of which in turn form the tendon unit. Primary, secondary, and tertiary bundles are surrounded by a sheath of connective tissue known as endotenon, which facilitates the gliding of bundles against one another during tendon movement. Endotenon is contiguous with epitenon, the fine layer of connective tissue that sheaths the tendon unit. Lying outside the epitenon and contiguous with it is a loose elastic connective tissue layer known as paratenon, which allows the tendon to move against neighbouring tissues. The tendon is attached to the bone by collagenous fibres (Sharpey fibres) that continue into the matrix of the bone.

The primary cell types of tendons are the spindle-shaped tenocytes (fibrocytes) and tenoblasts (fibroblasts). Tenocytes are mature tendon cells that are found throughout the tendon structure, typically anchored to collagen fibres. Tenoblasts are spindle-shaped immature tendon cells that give rise to tenocytes. Tenoblasts typically occur in clusters, free from collagen fibres. They are highly proliferative and are involved in the synthesis of collagen and other components of the extracellular matrix.

The composition of a tendon is similar to that of ligaments and aponeuroses.

A ligament is a tough fibrous band of connective tissue that serves to support the internal organs and hold bones together in proper articulation at the joints. A ligament is composed of dense fibrous bundles of collagenous fibres and spindle-shaped cells known as fibrocytes, with little ground substance (a gel-like component of the various connective tissues). Ligaments may be of two major types: white ligament is rich in collagenous fibres, which are sturdy and inelastic; and yellow ligament is rich in elastic fibres, which are quite tough even though they allow elastic movement. At joints, ligaments form a capsular sac that encloses the articulating bone ends and a lubricating membrane, the synovial membrane. Sometimes the structure includes a recess, or pouch, lined by synovial tissue; this is called a bursa. Other ligaments fasten around or across bone ends in bands, permitting varying degrees of movement, or act as tie pieces between bones (such as the ribs or the bones of the forearm), restricting inappropriate movement.

An aponeurosis is a flat sheet or ribbon of tendon-like material that anchors a muscle or connects it with the part that the muscle moves. The aponeurosis is composed of dense fibrous connective tissue containing fibroblasts (collagen-secreting spindle-shaped cells) and bundles of collagenous fibres in ordered arrays. Aponeuroses are structurally similar to tendons and ligaments.

In the United States alone, a very large number of anterior cruciate ligaments (ACL) are tom every year (approx. 200,000). A significant number of rotator cuff (approx. 50,000) and Achilles' tendons are also damaged (approx. 2,000). The number of torn or damaged tendon and ligaments has been increasing over time with the rise of participation in sports in the general population. Often the standard care is based on ligament reconstruction. Several replacement tissues can be envisaged using either grafts (auto, allo and xeno) or artificial materials.

Xenografts, ligaments from other animals, and allografts from cadaveric human tissue are possibilities that overcome the need to autologous tissues and avoid the risk of donor-site morbidity. However, their use poses several issues including risks of disease transmission, graft rejection and inflammation. Further, allografts and xenografts tend to be significantly weaker than native human tendons. Moreover, in the case of allografts, the supply is so small that the market demand can never be met from this source. Autograft tissues extracted from the patellar tendon, quadriceps tendonpatellar bone or the hamstring tendons are currently the most common sources of grafts for ACL reconstruction. Yet this therapy relies on the extraction of healthy tissue which implies risks of donor-site morbidity, and a long and painful recovery period.

The use of artificial prosthetic ligaments in the past as an alternative to autografts has brought about some improvements in existing reconstruction therapies. Some prior art materials which have been investigated are polyester, polytetrafluoroethylene and other fluoropolymers, carbon fibers, polyethylene, nylon and polystyrene. However none of these artificial ligaments have demonstrated positive long term results in vivo. Failures of previous devices mostly originate from mechanical failures, which include (i) rupture caused by wear, fatigue or severe loading in the knee and (ii) laxity in the joint after creep of the prosthetic ligament or loosening of the fixation element in the bone. There are also biocompatibility issues which can occur, which primarily manifest as immunogenic particulation leading to chronic synovitis. Due to high incidence of such problems, most if not all the previous artificial ligaments have been withdrawn from the commercial market.

Previous synthetic ligaments such as Goretex, Dacron and Leeds Keio all suffered from release of wear particles arising from local joint movement, leading to inflammatory responses (synovitis) and premature failure. Currently, out of the commercially available synthetic scaffolds for tendon or ligament repair, LARS® (Ligament Augmentation & Reconstruction System—see http://www.lars-ligaments.com/) is the only one being used clinically, though as a ligament augmentation device rather than as a complete ligament replacement.

There is a significant clinical need for readily available, off-the-shelf, implantable scaffolds. In particular, implantable synthetic ligament and tendon scaffolds for partial or full repair of ruptured or diseased tendons and ligaments. However, engineering a synthetic ligament or tendon scaffold is a significant challenge. In particular, to match tensile mechanical strength and stiffness of native load-bearing tendons, such as the shoulder rotator cuff and Achilles, and ligaments such as the anterior cruciate ligament. These challenges are made more difficult as the synthetic implantable scaffold must also provide conditions substantially equivalent to those found surrounding native tendons and ligaments, for example, hydrophilicity and equilibrium water content. Further still, re-injury and inflammation of joints treated with both biological and synthetic scaffolds are issues that still need to be addressed.

It is an object of the present invention to overcome or ameliorate one or more of the disadvantages of the prior art, or at least to provide a useful alternative.

### SUMMARY OF THE INVENTION

According to a first aspect, the present invention provides a synthetic implantable scaffold comprising:
a plurality of polymer fibres in contact with a composition comprising:
a hydrogel-forming polymer, and
a biocompatible ceramic material.

Preferably the synthetic implantable scaffold comprises tensile strength in the range 50 to 170 MPa. Preferably the synthetic implantable scaffold comprises a tensile modulus in the range of 500 to 2500 MPa.

Preferably the fibre volume fraction of the scaffold is between about 5-95%.

Preferably the composition constitutes between about 20-50 wt % of the synthetic implantable scaffold. Preferably the porosity of the scaffold is about 20 to 50 vol. %.

Preferably the plurality of polymer fibres comprises from 2 to 1000 individual fibres. Preferably the diameter of the individual polymer fibres is between about 1 to about 50 micrometers. Preferably the polymer fibres have a molecular mass between 1 and 8 million g/mol. Preferably the polymer fibres are formed from ultra-high molecular weight polyethylene (UHMWPE). Preferably the individual UHMWPE fibres is between about 2.5 to 5 GPa.

Preferably the plurality of individual polymer fibres is in the form of a bundle of fibres. Preferably the bundle of polymer fibres has a cross-sectional diameter of between about 150 to 1000 micrometers.

Preferably the synthetic implantable scaffold further comprises a plurality of bundles of individual polymer fibres. Preferably the plurality of bundles has a diameter of between about 1 to 10 mm. Preferably at least some of the plurality of polymer fibres and/or bundles are wound or twisted around other fibres or bundles to form a yarn or a braid.

In some embodiments, the implantable scaffold is in the form of a synthetic ligament replacement. Preferably the synthetic ligament is selected from the group consisting of: anterior-cruciate ligament, medial collateral ligament, lateral collateral ligament, posterior cruciate ligament, cricothyroid ligament, periodontal ligament, anterior sacroiliac ligament, posterior sacroiliac ligament, sacrotuberous ligament, inferior pubic ligament, superior pubic ligament, suspensory ligament of the penis, suspensory ligament of the breast, volar radiocarpal ligament, dorsal radiocarpal ligament, ulnar collateral ligament, and radial collateral ligament.

In some embodiments, the implantable scaffold is in the form of a synthetic tendon replacement. In this embodiment, the synthetic tendon may be selected from the group consisting of: rotator cuff tendon, elbow tendon, wrist tendon, hamstring tendon, patellar tendon, ankle tendon, and foot tendon.

In some embodiments, the hydrogel-forming polymer is polyvinyl alcohol (PVA), and the molecular weight of the PVA is between about 80,000 and about 100,000 g/mol.

Preferably the hydrogel-forming polymer is present in the composition at between about 5 wt % and about 25 wt %.

In some embodiments, the composition further comprises a cell adhesion promoter, wherein the cell adhesion promoter comprises gelatin. Preferably the gelatin is derived from collagen, and is optionally an irreversibly hydrolyzed form of collagen.

Preferably the concentration of gelatin in the composition is between about 0.1 wt % and about 10 wt %. In some embodiments, the ratio of hydrogel-forming polymer:gelatin is between 1:1 to 50:1 (weight %).

In some embodiments, the biocompatible ceramic material is Hardystonite (Ca₂ZnSi₂O₇) doped with Sr, Mg or Ba. Preferably the Hardystonite is strontium-doped Ca₂ZnSi₂O₇. Preferably the strontium-doped Hardystonite is present in the form of microparticles dispersed within the composition, wherein the microparticles have a diameter of between about 0.1 to 500 micrometers.

In some embodiments, the ratio of hydrogel-forming polymer:biocompatible ceramic material is between 0.5:1 to 10:1.

In some embodiments, the synthetic implantable scaffold has an equilibrium water content of between about 20 to about 80 wt %.

According to a second aspect, the present invention provides a method for preparing a synthetic implantable scaffold, the method comprising the steps of:
providing a plurality of polymer fibres;
providing a composition comprising: a hydrogel-forming polymer, and a biocompatible ceramic material; and
contacting the plurality of polymer fibres with the composition to thereby form said synthetic implantable scaffold.

In some embodiments, the method further comprises the step of providing from 2 to 1000 individual polymer fibres. Preferably the diameter of the individual polymer fibres is between about 1 to about 50 micrometers.

In some embodiments, the method further comprises the step of providing the plurality of individual polymer fibres in the form of a bundle of fibres, wherein the bundle of polymer fibres comprises a cross-sectional diameter between about 150 to 1000 micrometers.

In some embodiments, the method further comprises the step of providing a plurality of bundles of individual polymer fibres, wherein the plurality of bundles has a diameter of between about 1 to 10 mm.

In some embodiments, the method further comprises the step of winding or twisting at least some of the plurality of polymer fibres around other fibres to form a yarn or a braid.

In one preferred embodiment, the plurality of polymer fibres are uniaxially oriented and in the form of one or more bundles. In other preferred embodiments, the method comprises the step of impregnating the composition into the plurality of polymer fibres or the plurality of polymer fibres in the form of one or more bundles. It will be appreciated that impregnation of the composition substantially fills the voids between the polymer fibres and optionally any porosity within the fibres. Preferably any interstitial spaces can be filled with the composition, or alternatively the majority of the interstitial spaces are filled with the composition. In alternative embodiments the polymer fibres are substantially coated with the composition. In some embodiments, the fibres can be arranged in any suitable way to resemble or substantially resemble the fibrous microstructure of tendons and ligaments. In this embodiment, the collagen fibre arrangement may not necessarily be in a substantially uniaxial configuration.

In some embodiments, the polymer fibres have a molecular mass between 1 and 8 million g/mol. In some embodiments, the polymer fibres are formed from ultra-high molecular weight polyethylene (UHMWPE), wherein the strength of the UHMWPE fibre is between about 2.5 to 5 GPa.

In some embodiments, the implantable scaffold produced from the method is in the form of a synthetic ligament, wherein the synthetic ligament is selected from the group consisting of: anterior-cruciate ligament, medial collateral ligament, lateral collateral ligament, posterior cruciate ligament, cricothyroid ligament, periodontal ligament, anterior sacroiliac ligament, posterior sacroiliac ligament, sacrotuberous ligament, inferior pubic ligament, superior pubic ligament, suspensory ligament of the penis, suspensory ligament of the breast, volar radiocarpal ligament, dorsal radiocarpal ligament, ulnar collateral ligament, and radial collateral ligament.

In some embodiments, the implantable scaffold produced from the method is in the form of a synthetic tendon, wherein the synthetic tendon is selected from the group consisting of: rotator cuff tendon, elbow tendon, wrist tendon, hamstring tendon, patellar tendon, ankle tendon, and foot tendon.

In some embodiments, the method further comprises the step of providing the hydrogel-forming polymer at between about 5 wt % and about 25 wt %. Preferably the hydrogel-forming polymer is polyvinyl alcohol having a molecular weight between about 80,000 and about 100,000 g/mol.

In some embodiments, the method further comprises the step of providing a cell adhesion promoter, wherein the cell adhesion promoter comprises gelatin. Preferably the gelatin at a concentration between about 0.1 wt % and about 10 wt %.

In some embodiments, the method further comprises the step of providing the biocompatible ceramic material in the form of Hardystonite (Ca₂ZnSi₂O₇) doped with Sr, Mg or Ba. Preferably the Hardystonite is strontium-doped Ca₂ZnSi₂O₇. In some embodiments, the method further comprises the step of providing the strontium-doped Hardystonite in the form of microparticles dispersed within the composition, wherein the microparticles have a diameter of between about 0.1 to 500 micrometers.

In some embodiments, the method further comprises the step of providing the ratio of hydrogel-forming polymer:biocompatible ceramic material between 0.5:1 to 10:1.

In some embodiments, the method further comprises the step of pultruding the polymer fibres through a die thereby impregnating the composition into the plurality of polymer fibres. In some embodiments, the method further comprises the step of resting the synthetic implantable scaffold at about 20° C. for about 5 minutes after pultrusion. In some embodiments, the method further comprises the step of immersing the synthetic implantable scaffold in deionized water for a predetermined period of time and then freeze-drying.

According to a third aspect, the present invention provides a synthetic implantable scaffold prepared by the method according to the second aspect. In preferred embodiments the scaffold is a synthetic tendon or ligament.

According to a fourth aspect, the present invention provides a method for preparing a composition for use in preparing a synthetic implantable scaffold, the method comprising the steps of:
combining the following components: a hydrogel-forming polymer, biocompatible ceramic material, water and optionally acid; and
mixing said components to achieve homogeneous mixture, thereby to provide said composition.

In some embodiments, the method further comprises the step of providing the hydrogel-forming polymer in the form of polyvinyl alcohol having a molecular weight between about 80,000 and about 100,000 g/mol. Preferably the hydrogel-forming polymer is provided at between about 5 wt % and about 25 wt %.

In some embodiments, the method further comprises the step of providing a cell adhesion promoter, wherein the cell adhesion promoter comprises gelatine, wherein the gelatine is provided at a concentration between about 0.1 wt % and about 10 wt %.

In some embodiments, the method further comprises the step of providing the biocompatible ceramic material in the form of Hardystonite (Ca₂ZnSi₂O₇) doped with Sr, Mg or Ba. Preferably the Hardystonite is strontium-doped Ca₂ZnSi₂O₇.

In some embodiments, the method further comprises the step of providing the strontium-doped Hardystonite in the form of microparticles dispersed within the composition, wherein the microparticles have a diameter of between about 0.1 to 500 micrometers.

In some embodiments, the method further comprises the step of providing the ratio of hydrogel-forming polymer:biocompatible ceramic material between 0.5:1 to 10:1.

In some embodiments, the method further comprises the step of adding acid such the pH is about 7.0 to 7.5.

In some embodiments, the method further comprises the step of heating the mixture to between about 70 to 95° C.

In some preferred embodiments, the hydrogel-forming polymer is PVA. In some preferred embodiments the biocompatible ceramic material is Sr—HT. In some preferred embodiments, the acid is hydrochloric acid for neutralizing the slight alkalinity of the bioactive ceramic material. The skilled person will appreciate that other acids can be used instead of, or in addition to, hydrochloric acid. The method preferably further comprises adding a cell adhesion promoter, such as gelatin. Without wishing to be bound by theory, it is contemplated that the hydrogel-forming polymer assists in mimicking the fibre-ECM hierarchical structure of native tendons or ligaments. It is also contemplated that the hydrogel-forming polymer provides a porous structure for retention of water within the scaffold in vivo. It is further contemplated that the hydrogel-forming polymer assists to reduce friction when the scaffold is in situ. Yet further still, it is contemplated that gelatin assists with cell adhesion, and the biocompatible ceramic material assists in promoting cell activity in vivo.

As disclosed herein, there is provided use of a composition produced by the method of the fourth aspect for preparing a synthetic implantable scaffold.

According to a fifth aspect, the present invention provides use of the implantable scaffold according to the first aspect for partial or full tendon or ligament repair.

According to a sixth aspect, the present invention provides a method of partial or full tendon or ligament repair in a patient comprising implantation of a synthetic implantable scaffold according to the first aspect.

According to a further aspect, the present invention provides a synthetic implantable scaffold of the invention for use in partial or full tendon or ligament repair in a patient.

According to a seventh aspect, the present invention provides use of a synthetic implantable scaffold according to the first aspect in the manufacture of a medicament for partial or full tendon or ligament repair in a patient.

According to a ninth aspect, the present invention provides a composition comprising a hydrogel-forming polymer; cell adhesion promoter; and biocompatible ceramic material for use in partial or full tendon or ligament repair in combination with a plurality of polymer fibres. Preferably the hydrogel-forming polymer is PVA. Preferably the biocompatible ceramic material is Sr—HT. Preferably the cell adhesion promoter is gelatin.

According to a tenth aspect, the present invention provides use of a composition comprising a hydrogel-forming polymer; cell adhesion promoter, and biocompatible ceramic material in the manufacture of synthetic tendon or ligament scaffold. Preferably the hydrogel-forming polymer is PVA. Preferably the biocompatible ceramic material is Sr—HT. Preferably the cell adhesion promoter is gelatin.

According to an eleventh aspect, the present invention provides use of a composition comprising a hydrogel-forming polymer; cell adhesion promoter; and biocompatible ceramic material in the manufacture of synthetic tendon or ligament scaffold for partial or full tendon or ligament repair in combination with a plurality of polymer fibres. Preferably the hydrogel-forming polymer is PVA. Preferably the biocompatible ceramic material is Sr—HT. Preferably the cell adhesion promoter is gelatin.

According to a twelfth aspect, the present invention provides a synthetic tendon or ligament comprising a plurality of the synthetic tendon or ligament scaffolds of the invention.

According to a thirteenth aspect, the present invention provides use of a plurality of the synthetic tendon or ligament scaffolds in the manufacture of a prosthesis for partial or full tendon or ligament repair.

According to a fourteenth aspect, the present invention provides a prosthesis comprising a plurality of the synthetic tendon or ligament scaffolds of the invention for partial or full tendon or ligament repair.

### BRIEF DESCRIPTION OF THE DRAWINGS

Some embodiments of the invention will now be described, by way of example only, with reference to the accompanying drawings in which:

FIG. 1 shows a suitable method of production of the scaffold of the invention, i.e. a pultrusion method is shown in which hot hydrogel solution is injected into a bundle of UHMWPE fibres which are then drawn out through a (4 mm) diameter outlet.

FIG. 2 shows a scanning electron (SEM) image of the UHMWPE+hydrogel composition, with energy dispersive X-ray spectroscopy analysis showing UHMWPE fibres, a PVA/gelatin hydrogel coating the UHMWPE fibres and fibrils interconnecting the UHMWPE fibres, and Sr—HT microparticles (circled in the figure). Scale bar 250 micrometers.

FIG. 3 shows representative stress-strain curves for UHMWPE, UHMWPE with a PVA hydrogel (PVA-UHMWPE), PVA+gelatin (PG-UHMWPE) and PVA+gelatin+Sr—HT (PSG-UHMWPE).

FIG. 4 is a bar chart showing tensile strength for UHMWPE, compared to UHMWPE with a PVA hydrogel (PVA-UHMWPE), PVA+gelatin (PG-UHMWPE), and PVA+gelatin+Sr—HT (PSG-UHMWPE).

FIG. 5 is a bar chart, showing tensile moduli (both toe and linear region) for UHMWPE, compared to UHMWPE with a PVA hydrogel (PVA-UHMWPE), PVA+gelatin (PG-UHMWPE), and PVA+gelatin+Sr—HT (PSG-UHMWPE).

FIG. 6 is a bar chart showing the equilibrium water content of the UHMWPE, UHMWPE with a PVA hydrogel (PVA-UHMWPE), PVA+gelatin (PG-UHMWPE) and PVA+gelatin+Sr—HT (PSG-UHMWPE) scaffolds.

FIG. 7 is a bar chart plotting absorbance at 490 nm on day 3 and day 7 for three oMSC cell proliferation assays.

FIG. 8 is an SEM image, which shows cells (indicated with arrows) attached onto the surface of a scaffold of the invention (PSG-UHMWPE) cultured for 24 hours.

FIG. 9 are SEM-EDS overlay images of UHMWPE fibres coated with PVA (top row); UHMWPE fibres coated with gelatin (middle row); and the scaffold of the invention (lower row). Scale bar represents 100 μm. It can be seen that the individual fibres are arranged into primary bundles, and there are a plurality of secondary bundles which comprise the scaffold.

FIG. 10a) is a photograph of the lyophilized scaffolds in each group discussed herein; and b) is a light microscopy image of representative hydrated scaffold specimen. Scale bar=2.0 mm

FIG. 11 are scanning electron microscopy images of oMSCs cultured for 24 h on a) UHMWPE with a PVA hydrogel (PVA-UHMWPE); b) PVA+gelatin (PG-UHMWPE); and c) PVA+gelatin+Sr—HT (PSG-UHMWPE) scaffolds. (Note FIG. 8 is an expanded view of FIG. 11c).

FIG. 12 shows intraoperative photographs of the right Achilles tendon of an animal of the control group in the in vivo study: (a) prepared Achilles tendon before tenotomy; (b) dissected tendon; and (c) sutured tendon stumps; and also shows (d) a schematic illustration of the primary tendon suture (Kirchmayr-Kessler-suture).

FIG. 13 shows intraoperative photographs of the right Achilles tendon of an animal of the scaffold group in the in vivo study: (a) prepared tendon before creating the defect; (b) 5 mm tendon defect; and (c) suturing of the scaffold to the proximal end of the tendon; and also shows (d) a schematic illustration of the modified suture used.

FIG. 14 shows the sectioning used in the in vivo study for the control group and the scaffold group.

FIG. 15 shows the results of the macroscopic scoring of native tendon, control and scaffold groups in the in vivo study according to Stoll et al. (median=long dash and range=short dashes).

FIG. 16 shows the maximum force (in N) of the implant and control-group as well as of native tendon and non-implanted scaffold material in the in vivo study (median=long dash and range=short dash).

FIG. 17 shows the stiffness κ (in N/mm) in cycle 1 and cycle 5 of the implant and control-group in the in vivo study as well as that of native tendon and non-implanted scaffold material (median with range).

FIG. 18 shows the Young's modulus (in Mpa) of the implant and control-group in the in vivo study as well as of native tendon and non-implanted scaffold material (median with range).

FIG. 19 shows in vivo integration of a scaffold of the invention into surrounding native tendon tissue.

FIG. 20 shows a longitudinal section of an unstained non-implanted scaffold under polarized light, illustrating fibre arrangement of the device used for the in vivo study.

FIG. 21 shows a cross-section of an unstained non-implanted scaffold under polarized light, illustrating fibre arrangement of the device used for the in vivo study.

### DEFINITIONS

In describing and claiming the present invention, the following terminology will be used in accordance with the definitions set out below. It is also to be understood that the terminology used herein is for the purpose of describing particular embodiments of the invention only and is not intended to be limiting. Unless defined otherwise, all technical and scientific terms used herein have the same meaning as commonly understood by one having ordinary skill in the art to which the invention pertains.

The recitation of a numerical range using endpoints includes all numbers subsumed within that range (e.g., 1 to 5 includes 1, 1.5, 2, 2.75, 3, 3.80, 4, 5, etc.).

The terms “preferred”, “preferably” and “suitably” refer to embodiments of the invention that may afford certain benefits, under certain circumstances. However, other embodiments may also be preferred, under the same or other circumstances. Furthermore, the recitation of one or more suitable embodiments does not imply that other embodiments are not useful, and is not intended to exclude other embodiments from the scope of the invention.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”.

Other than in the operating examples, or where otherwise indicated, all numbers expressing quantities of ingredients or reaction conditions used herein are to be understood as modified in all instances by the term “about”. The examples are not intended to limit the scope of the invention. In what follows, or where otherwise indicated, “%” will mean “weight %”, “ratio” will mean “weight ratio” and “parts” will mean “weight parts”.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviations found in their respective testing measurements.

As used herein, the term ‘implantable scaffold’ means a synthetic implantable scaffold, which is preferably in the form of a synthetic ligament or tendon that can be installed in vivo to repair, replace or augment a ruptured or diseased ligament or tendon. The skilled person will appreciate that replacement of a ligament or tendon will comprise excision of the pre-existing ruptured or diseased tissue and complete replacement with the synthetic implantable scaffold of the invention (‘full replacement’). It will also be appreciated that under some circumstances only a portion of the pre-existing tissue may require excision, and that only the excised portion will need to be replaced with the synthetic implantable scaffold of the invention (‘partial replacement’). Alternatively, it may be that some or all of the pre-existing tissue requires excision, but the surgeon decides that none of the ruptured or diseased tissue is excised and that the synthetic implantable scaffold of the invention is used to augment the existing tissue (‘augmentation’). Variations of these combinations will be apparent to the skilled person. The term “patient” generally refers to humans or other mammals.

As used herein, ‘implantable’ or ‘suitable for implantation’ means surgically appropriate for insertion into the body of a host, e.g., biocompatible, or having the design and physical properties set forth in more detail below. Preferably, the implantable scaffold is designed and dimensioned to function in the surgical repair, augmentation, or replacement of damaged tissue, such as, e.g., a rotator cuff, including fixation of tendon-to-bone.

As used herein, when the implantable scaffold of the invention is used in the form of a synthetic ligament, the synthetic ligament is selected from the group consisting of: anterior-cruciate ligament, medial collateral ligament, lateral collateral ligament, posterior cruciate ligament, cricothyroid ligament, periodontal ligament, anterior sacroiliac ligament, posterior sacroiliac ligament, sacrotuberous ligament, inferior pubic ligament, superior pubic ligament, suspensory ligament of the penis, suspensory ligament of the breast, volar radiocarpal ligament, dorsal radiocarpal ligament, ulnar collateral ligament, and radial collateral ligament.

As used herein, when the implantable scaffold of the invention is used in the form of a synthetic tendon, the tendon is selected from the group consisting of: rotator cuff tendon, elbow tendon, wrist tendon, hamstring tendon, patellar tendon, ankle tendon, and foot tendon. In particular, the tendon is the supra-spinatus tendon, the Achilles tendon, or the patellar tendon.

As used herein, ‘biomimetic’ shall mean a resemblance of a synthesized material to a substance that occurs naturally in a human body and which is not rejected by (e.g., does not cause an adverse reaction in) the human body. When used in connection with the term ‘implantable scaffold’, biomimetic means that the implantable scaffold is biologically inert (i.e., will not cause an immune response/rejection) and is designed to resemble a structure that occurs naturally in a mammalian, e.g., human, body.

As used herein, ‘synthetic’ scaffold means that the scaffold is composed of man-made material, such as synthetic polymer, or a synthetic ceramic, but it does not preclude further treatment with material of biological or natural origin, such as seeding with appropriate cell types, e.g., seeding with osteoblasts, osteoblast-like cells, and/or stem cells, or treating with a medicament, e.g., anti-infectives, antibiotics, bisphosphonate, hormones, analgesics, antiinflammatory agents, growth factors, angiogenic factors, chemotherapeutic agents, anti-rejection agents, and RGD peptides.

As used herein, ‘hydrogel’ shall mean any colloid in which the particles are in the external or dispersion phase and water is in the internal or dispersed phase. PVA is an abbreviation for polyvinyl alcohol.

As used herein, ‘polymer fibres’ shall mean fibres which are formed from naturally-occurring or man-made polymers. Preferred fibres are formed form polymers which are inert and have high molecular weight, or preferably ultra high molecular weight. Preferred polymers are not biodegradeable. Preferred molecular mass is between 1 and 8 million g/mol. Preferred diameters of individual polymer fibres match the ranges reported for individual collagen fibres (e.g. 5 to 30 micrometers). Preferably the polymer fibres are oriented in such a way (i.e., aligned or unaligned) so as to mimic the natural architecture of the tissue to be repaired.

UHMWPE is an abbreviation for ultra-high molecular weight polyethylene. UHMWPE has extremely long chains of polyethylene which all align in the same direction, and typically has a molecular mass usually between 3.5 and 7.5 million g/mol. UHMWPE is a very tough material, with one of the highest impact strengths of any thermoplastic polymer. When formed into fibres, the polymer chains can attain a parallel orientation greater than 95% and a level of crystallinity from 39% to 75%.

ECM is an abbreviation for extracellular matrix.

Sr—HT means Sr-doped Hardystonite. This may also be referred to as strontium calcium zinc silicate.

PVA-UHMWPE means PVA hydrogel and UHMWPE fibres.

PG-UHWMPE means PVA hydrogel incorporated with gelatin and UHMWPE fibres.

PSG-UHMWPE means PVA hydrogel incorporated with gelatin and Sr—HT and UHMWPE fibres.

MSC proliferation assay is a term of the art, with which the skilled person would be familiar. MSC is an abbreviation for Mesenchymal Stem Cell. oMSC relates to ovine MSC.

SEM is an abbreviation for scanning electron microscopy.

The term ‘prosthesis’ is generally used to describe an artificial device that replaces a missing body part, which may be lost through trauma, disease, or congenital conditions. The term ‘scaffold’ generally refers to materials that have been engineered to cause desirable cellular interactions to contribute to the formation of new functional tissues for medical purposes. For the purposes of the present invention, the terms prosthesis and scaffold are used interchangeably.

The ACL prosthesis is formed of a plurality of independent fibres. Individual fibres have small diameters in order to limit the bending strain. Multiple fibres operate together to provide the necessary strength for the ACL prosthesis.

### DETAILED DESCRIPTION OF THE INVENTION

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims. One skilled in the art will recognize many methods and materials similar or equivalent to those described herein, which could be used in the practice of the present invention. The present invention is in no way limited to the methods and materials described.

As disclosed herein, there is provided a synthetic implantable scaffold comprising a plurality of polymer fibres in contact with a composition, wherein the composition comprises a hydrogel-forming polymer and a biocompatible ceramic material.

In one preferred embodiment, the present invention provides a synthetic tendon or ligament scaffold comprising:
a plurality of polymer fibres in the form of one or more bundles,
wherein the one or more bundles are impregnated with an impregnation composition comprising:
   a hydrogel-forming polymer, and
   a biocompatible ceramic material.

With reference to the above preferred embodiment, the combination of polymer fibres in the form of a bundle and impregnation thereof with an impregnation composition comprising a hydrogel-forming polymer and a biocompatible ceramic material provides a new synthetic tendon or ligament scaffold with many advantages compared to prior art devices. For example, the scaffold of the invention provides high equilibrium water content, and simultaneously provides the ability to support cell adhesion and proliferation exhibited in the tendon extracellular matrix (ECM). Additionally, the scaffold of the invention advantageously provides the needed hydrophilicity to allow for adhesion of water soluble proteins, and to prevent fibrous adhesion with neighbouring tissue. These advantages will be discussed further in the following.

In one embodiment, the synthetic tendon or ligament scaffold of the present invention may have an ultimate tensile strength (σᵤₜₛ) and modulus (E) in the reported range of Achilles' tendon. For example, the synthetic tendon or ligament scaffold of the present invention may be configured to provide tensile strength in the range 50 to 170 MPa, for example 50 to about 70, 70 to about 90, 90 to about 110, 110 to about 130, 130 to about 150, or 150 to about 170 MPa. Additionally, the tendon or ligament scaffold of the invention may be configured to provide a tensile modulus in the range of 500 to 1750 MPa, for example 500 to 750, 750 to 1000, 1000 to 1250, 1250 to 1500, or 1500 to 1750 MPa.

In other embodiments, the synthetic tendon or ligament scaffold of the present invention may be configured to provide an ultimate tensile strength (σᵤₜₛ) and tensile modulus (E) in the reported range of the shoulder rotator cuff. For example tensile strength at about 20 MPa, and a tensile modulus of 50 to 70, 70 to 90, 90 to 110, 110 to 130, 130 to 150, or 150 to 170 MPa.

In other embodiments, the synthetic tendon or ligament scaffold of the present invention may be configured to provide an ultimate tensile strength (Oᵤ) and tensile modulus (E) in the reported range of other ligaments, such as the anterior cruciate ligament. For example tensile strength at about 25 MPa, and a tensile modulus of around 110 MPa.

Advantageously, the synthetic implantable scaffold of the present invention can be formulated to have high equilibrium water content, which is similar to that of a native tendon or ligament. In one embodiment the synthetic implantable scaffold of the present invention may have a water content of about 70 wt %. In other examples, the water content is between about 20 to about 80 wt %, or about 60 to 90 wt %, or about 65 to 75 wt %, or about 20 to 50 wt %, or about 40 to 75 wt %, such as about 25, 30, 35, 40, 45, 50, 60, 65, 70, or 75 wt %.

In one embodiment, the fibre volume fraction of the scaffold is between about 5-95%. In some embodiments, the composition fraction in the scaffold is within about 20-50 wt %. It will be appreciated that there can be some porosity within the scaffold, which could be in the range of around 20 to 50 vol %. In another embodiment, the osmolality of the scaffold is balanced via the hydrogel fraction to equilibrate with the native tissue being mimicked.

### Polymer Fibres

The implantable scaffold of the invention includes a plurality of polymer fibres, which may be formed from naturally-occurring or man-made polymers. Preferred polymers are inert and have high molecular weight, or more preferably ultra high molecular weight, are biocompatible, but not substantially biodegradeable. The present invention also contemplates mixtures of polymers or co-polymers used to fabricate the polymer fibres.

In advantageous embodiments according to the invention, the polymer is selected from the group consisting of polyethylene (PE), polypropylene (PP), polyamides (PA), polycarbonates (PC), polyurethanes (PU), polyurethane urea, polyesters like polyethylene terephtalate (PET), polyfluoropolymers like polytetrafluoroethylene (PTFE), polyacrylates like polymethyl methacrylate (PMMA), polyethylene glycol (PEG), and from blends or copolymers obtained with polymers from this group. Accordingly, the polymer may be polyethylene (PE), polypropylene (PP), a polyamide (PA), a polycarbonate (PC), a polyurethane (PU), a polyurethane urea, polyester, including polyethylene terephtalate (PET), a polyfluoropolymer such as polytetrafluoroethylene (PTFE), a polyacrylate such as polymethyl methacrylate (PMMA), a polyethylene glycol (PEG), or a blend or copolymer of any two or more of these polymers. Suitable polymer fibres are ultra high molecular weight polyethylene fibres (UHMWPE). In other embodiments, the polymer fibre bundle may also contain other types of biocompatible fibers assembled with the polymer fibers, for example biocompatible metal fibers like titanium and titanium alloy fibers. Other suitable polymeric fibers are polyethylene teraphtalate (polyester), polyamide (NYLON®), or aramid (KEVLAR®). Resorbable fibers can additionally be used, e.g. those based on poly lactic acid or polyglycolic acid.

In some embodiments, the preferred molecular mass of the polymer is between 500,000 and 1 million g/mol. In other embodiments, the preferred molecular mass of the polymer is between 1 and 8 million g/mol, or between 3.5 and 7.5 million g/mol. Preferably the tensile strength of the polymer for use in the present invention is about 1, 2.0, 2.5, 3, 3.5, 4, 4.5, or 5 GPa. For simplicity, polymer fibres having a tensile strength of at least 2.5 GPa are hereinafter referred to as high strength fibres.

Preferably the polymer is UHMWPE. UHMWPE is synthesized from monomers of ethylene, which are bonded together to form the base polyethylene product. These molecules are several orders of magnitude longer than those of familiar high-density polyethylene (HDPE) due to a synthesis process based on metallocene catalysts, resulting in UHMWPE molecules typically having 100,000 to 250,000 monomer units per molecule each compared to HDPE's 700 to 1,800 monomers. UHMWPE has a molecular mass usually between 3.5 and 7.5 million, and is typically processed variously by compression molding, ram extrusion, gel spinning, and sintering. UHMWPE has extremely long chains of polyethylene which all align in the same direction. UHMWPE fibres have high tensile strength and are bioinert. UHMWPE is also a very tough material, with one of the highest impact strengths of any thermoplastic polymer. When formed to fibres, the polymer chains can attain a parallel orientation greater than 95% and a level of crystallinity from 39% to 75%. Suitably, highly cross-linked UHMWPE may be used (with gamma or electron beam radiation) and then thermally processed to improve oxidation resistance.

In the context of polymer fibres, ‘plurality’ may refer to from 2 to 1000 polymer fibres, e.g. 2 to 5, 5 to 10, 10 to 15, 15 to 20, 20 to 25, 25 to 30, 30 to 35, 35 to 40, 40 to 45, 45 to 50, 50 to 55, 55 to 60, 60 to 65, 65 to 70, 70 to 75, 75 to 80, 80 to 85, 85 to 90, 90 to 95, 95 to 100, 100 to 200, 200 to 300, 300 to 400, 400 to 500, 500 to 600, 600 to 700, 700 to 800, 800 to 900, or 900 to 1000 fibres. The number of fibres can be chosen to suit the application and the required mechanical properties. The skilled person will appreciate that the number of fibres used in the scaffold of the invention can also depend on the fibre thickness. For example, use of relatively thicker fibres can mean that relatively fewer fibres are required in the primary fibre bundle, and vice versa. Preferably the plurality of polymer fibres is in the form of a bundle. The plurality of polymer fibres may be the number of fibres in a primary bundle. There may be a single primary bundle in the synthetic implantable scaffold of the invention, or there may be two or more primary bundles in the scaffold, each primary bundle comprising a plurality of polymer fibres.

Accordingly, the present invention contemplates a plurality of bundles of fibres, i.e. bundles of bundles. To explain, the present invention contemplates a bundle of fibres (or a ‘primary bundle’) and secondary bundles, which are bundles of primary bundles. Further, tertiary bundles of secondary bundles are also contemplated, as are quatemary bundles of tertiary bundles, etc. The secondary (and tertiary, and quatemary, etc) bundles may comprise from 2 to 100 primary bundles, e.g. 2 to 5, 5 to 10, 10 to 15, 15 to 20, 20 to 25, 25 to 30, 30 to 35, 35 to 40, 40 to 45, 45 to 50, 50 to 55, 55 to 60, 60 to 65, 65 to 70, 70 to 75, 75 to 80, 80 to 85, 85 to 90, 90 to 95, or 95 to 100. The number of bundles can be chosen to suit the application and can depend on the total number of individual fibres required, and the cross-sectional diameter of the individual fibres being used.

The synthetic implantable scaffold of the invention preferably comprises an elongate bundle of fibres. In the elongate bundle, a single fibre may be wound from end to end of the elongate bundle. Suitably the plurality of polymer fibres are longitudinally aligned in a parallel configuration, or in a substantially parallel configuration, in the scaffold. However, in other embodiments one or more of the plurality of fibres are wound or twisted around other fibres to form a yarn, and can include braids. In still further embodiments, the arrangement of the fibres can be such as to mimic non-parallel fibre arrangements found in natural certain tendons and/or ligaments, where the collagen fibre arrangement is not primarily uniaxial. In such arrangements, the fibres may be longitudinally aligned but non-parallel, or may be non-longitudinally aligned in whole or in part. The skilled person will appreciate the other configurations that fall within the purview of the invention.

Suitably, the diameters of the individual polymer fibres match the ranges reported for individual collagen fibres (5 to 30 micrometers). For example, the diameters may be about 1 to about 50, about 2 to about 40, about 5 to about 30, about 10 to about 30 or about 20 to about 30 micrometers, particularly 20 to 30 micrometers. In some embodiments, the fibres are all the same approximate diameter (e.g. the same diameter within 10%, such as within 5%). In other embodiments, the fibres may have the same or different diameters within a defined range (e.g. 20 to 30 micrometers). In other embodiments the fibres are chosen to have a plurality of different diameters, or may be in the form of a tape or ribbon. In some embodiments, the fibres are all the same cross sectional shape, which is suitably circular, and in other embodiments the fibres are chosen to have different cross sectional shapes. In some embodiments, the fibres have a hollow core (lumen structure), and in other embodiments the fibres are fabricated to have substantial surface porosity or roughness. In yet further embodiments, the molecular weight of the polymer comprising the fibre is chosen to obtain a fibre bundle having pre-determined mechanical properties. The skilled person will appreciate that fibre diameter, number of fibres, fibre cross-sectional shape, fibre surface roughness, and polymer type can be chosen to suit the intended application of the synthetic tendon or ligament scaffold of the invention.

Preferred diameters of individual polymer fibres match the ranges reported for individual collagen fibres (e.g. 5 to 30 micrometers). Preferably the polymer fibres are oriented in such a way (i.e., aligned or unaligned) so as to mimic the natural architecture of the tissue to be repaired.

Suitably the primary, secondary, and/or tertiary, etc bundles are configured to comprise a cross-sectional diameter similar to the cross-sectional diameter of fascicles (150 to 1000 micrometers), for example, about 150 to about 1000, about 900, about 800, about 700, about 600, about 500, about 400 or about 300 micrometers, or about 200 to about 1000, about 900, about 800, about 700, about 600, about 500, about 400 or about 300 micrometers, particularly 200 to 300 micrometers.

Suitably the plurality of fibres are formed from a polymer such that the resulting implantable scaffold may have a tensile strength ranging from 20-40 MPa. It will be appreciated that the number of fibres can be chosen to suit the application and tailored to suit the required mechanical properties of the implantable scaffold. For example, yield strengths for the implantable scaffold between 50 and 120 MPa are preferred. Preferably the yield strain is between 5 and 15%, tensile modulus (30 MPa; linear) between 500 and 2500 MPa, and tensile modulus (5 MPa; toe) between 500 and 1000 MPa. The tensile strength of the preferred UHMWPE fibre for use in the present invention is about 2.5, 3, 3.5, 4, 4.5, or 5 GPa. For simplicity, the UHMWPE fibres having a tensile strength of at least 2.5 GPa are hereinafter referred to as high strength UHMWPE fibres.

Suitably the synthetic implantable scaffold of the invention is configured to have an ultimate tensile strength between 40-100 MPa, e.g. 45-90 MPa and in particular, between 50-85 MPa. In one embodiment, the scaffold is configured by selection of the number and properties of the fibre.

In one embodiment, a 2 mm diameter synthetic scaffold of the invention can be configured, which lies within the typical range for naturally occurring tertiary fibre bundles (typically 1000 to 3000 micrometers). The 2 mm diameter synthetic scaffold can be formed of a bundle of individual fibres (a primary bundle), or a secondary bundle (of primary bundles), or a tertiary bundle (of secondary bundles), etc. It is contemplated that a plurality of such 2 mm diameter synthetic scaffolds could be joined together to form a full tendon replacement.

For example, in the case of an artificial ligament for repairing a human knee ligament, the diameter of the synthetic tendon or ligament scaffold is between 2 mm and 20 mm and may be in the form of a tape in cross section, at least in a portion of its length. In one embodiment the diameter may be between 5 mm and 10 mm. In another embodiment the diameter of the synthetic tendon or ligament scaffold may be from about 2 mm to about 10 mm, such as from about 2 mm to about 6 mm, e.g. about 4 mm.

Suitably the length of the scaffold is also similar to the length of the natural ligament. For the case of the human knee ligament, it is between 0.5 cm and 5 cm and the length of the whole ligament comprising the median part and the end parts is between 5 cm and 25 cm, advantageously between 10 cm and 20 cm, more advantageously is about 15 cm.

In some embodiments, the fibres are coated with healing promoters such as thrombosis inhibitors, fibrinolytic agents, vasodilator substances, anti-inflammatory agents, cell proliferation inhibitors, and inhibitors of matrix elaboration or expression; examples of such substances are provided in U.S. Pat. No. 6,162,537, to Solutia Inc. The present invention also contemplates using a polymer coating, (e.g., a resorbable polymer) in conjunction with a healing promoter to coat the fibres.

### Composition

As disclosed herein, there is provided a synthetic implantable scaffold comprising a plurality of polymer fibres in contact with a composition, wherein the composition comprises a hydrogel-forming polymer and a biocompatible ceramic material. In some embodiments, the composition is an impregnation composition, in that it impregnates the polymer fibres to form the synthetic implantable scaffold.

### Hydrogel

The composition described herein comprises a hydrogel-forming polymer. The composition is thus suitably in the form of a hydrogel. The preferred hydrogel of the invention is injectable and can be contacted with or impregnated through a densely packed fibrous structure, and retains its water-retaining capabilities, as well as being able to withstand significant tensile, compressive and shear strain. Preferably the hydrogel possesses sufficient initial viscosity, and sets relatively quickly so that the hydrogel does not leak out between the fibres of the scaffold during the contacting or impregnation procedure.

The present invention addresses the problem of diffusion of lubricating fluid out of the initial implant by incorporating a composition comprising a hydrogel-forming polymer (where suitably the composition is a hydrogel composition) that is able to retain its water content. As the hydrogel remains in the scaffold in vivo, water can diffuse in and out of the scaffold, effectively meaning that the hydrogel acts as a lubricant.

As the skilled person is aware, hydrogels are hydrophilic polymer networks that can absorb water and swell without dissolving, at least temporarily. Depending on the physicochemical properties, levels of water absorption can vary greatly from about 10% to a thousand times their dry weight. The hydrogels of the invention retain substantial water content, and suitably comprise a molecular structure very similar to living tissue. Suitably the hydrogels of the invention are biocompatible, and impart some lubricity and elasticity.

A suitable hydrogel-forming polymer is polyvinyl alcohol (PVA). PVA-based hydrogel has been surprisingly found to provide a porous architecture for retention of water molecules, and to impart low friction. In particular, PVA-based hydrogels mimic the ECM structure of native tendons and ligaments. The molecular weight of the PVA is suitably between about 80,000 and about 100,000 g/mol, e.g. between about 89,000 and about 98,000 g/mol. The PVA is suitably physically crosslinked in the presence of polyethylene glycol (see U.S. Pat. No. 7,776,352 B2 to Ruberti and Braithwaite incorporated herein by reference). Suitably, the PVA-based hydrogel does not contain copolymers. Suitably, the PVA-based hydrogel does not degrade over time. For example, the PVA-based hydrogel may be essentially non-degradable in normal physiological environments and therefore may remain substantially in vivo during the lifetime of a prospective patient.

The equilibrium water content for a PVA-based hydrogel may be about 1500%, or between about 500% and about 2000%, or between about 500% and about 1500%, or between about 500% and about 1000%, or between about 750% and about 1250%, or between about 1000% and 2000%, or between about 1200% and 1800%, or between about 100% and 1500%, or between about 1500 and 2000%, or of at least 500%, or of at least 1000%, or of at least 1200%, or of about 500%, 600%, 700%, 800%, 900%, 1000%, 1100%, 1200%, 1300%, 1400%, 1500%, 1600%, 1700%, 1800%, 1900% or 2000%. For example, in certain embodiments, the PVA-based hydrogel (PVA-UHMWPE) has an equilibrium water content of about 1500%, the PVA-based hydrogel (PG-UHMWPE) has an equilibrium water content of about 1200%, and/or the PVA-based hydrogel (PSG-UHMWPE) has an equilibrium water content of about 600%. Lyophilized PVA-based hydrogels suitable for the present invention typically have pore sizes between 5 to 40 micrometers.

It will be appreciated, however, that other non-PVA-based hydrogels may be suitable alternatives to PVA-based hydrogels. Non-PVA-based hydrogels may have equilibrium water contents of between about 500% and about 1500% and/or pore sizes between 5 to 40 micrometers when lyophilized.

The amount of composition comprising a hydrogel-forming polymer present in the scaffold relative to the plurality of fibres (or fibre bundle(s)) may vary depending on the number of fibres in the scaffold and the diameter of those fibres or fibre bundle(s). Where the plurality of fibres and/or fibre bundle(s) are substantially elongate or longitudinally aligned, and composition comprising a hydrogel-forming polymer is contacted with or impregnated into the fibres in a pultrusion process, the amount of composition will depend on the number of fibres being pulled through and the opening diameter. In one embodiment of the scaffold reported herein, the ratio of the dry weight of the hydrogel-forming polymer composition to fibre bundle is suitably between about 1 to 5 and 1 to 20, most suitably about 1 to 10.

Suitably the concentration of hydrogel-forming polymer in the composition is between about 5 wt % and about 25 wt %. For example, the concentration may be between about 5 wt % and about 20 wt %, between about 10 wt % and about 15 wt %, e.g. about 10, about 11, about 12, about 13, about 14, or about 15 wt %, e.g. about 13.5 wt %.

The hydrogel-forming polymer is capable of forming a hydrogel. The scaffold of the invention may thus be formed when the heated hydrogel-forming polymer composition is contacted with, or impregnated into, the plurality of polymer fibres and upon cooling, forms a hydrogel. The composition comprising a hydrogel-forming polymer may require a physical cross-linking agent, such as poly(ethylene glycol), e.g., PEG400, to assist in hydrogel formation. The physical cross-linking agent may be removed prior to use of the scaffold in vivo. In some embodiments, the composition may require a chemical cross-linking agent. The skilled person will understand that physical and/or chemical cross-linking agents may be selected according to the particular hydrogel-forming polymer(s) present in the composition.

### Biocompatible Ceramic Material

The scaffolds of the invention comprise biocompatible ceramic materials within the hydrogel, and are expected to provide a significant improvement in long-term performance of the scaffolds of the invention.

One suitable biocompatible ceramic material is Hardystonite (Ca₂ZnSi₂O₇) doped with Sr, Mg or Ba, as described in International PCT Publication No. WO 2010/003191, which is incorporated herein in its entirety.

One suitable doped Hardystonite is strontium-doped Ca₂ZnSi₂O₇ (Sr—HT). The molecular formula of Sr—HT is SrₓCa₍₂₋ₓ₎ZnSi₂O₇, wherein x lies between 0.05 to 0.9. Suitably x=0.1. Thus suitably the Sr—HT of the present invention is represented by the molecular formula Sr_{0.1}Ca_{1.9}ZnSi₂O₇. Alternatively x is 0.15, 0.2, 0.25, 0.3, 0.35, 0.4, 0.45, 0.5, 0.55, 0.6, 0.65, 0.7, 0.75, 0.8, 0.85, or 0.9, or between 0.05 and 0.15, or between 0.1 and 0.4, or between 0.3 and 0.7, or between 0.05 and 0.5, or between 0.5 and 0.9.

Suitably the Sr:Ca ratio is between about 0.025 to 0.85. For example the Sr:Ca ratio may have a value of 0.025, 0.05, 0.075, 0.1, 0.125, 0.15, 0.175, 0.2, 0.225, 0.25, 0.275, 0.3, 0.325, 0.35, 0.375, 0.4, 0.425, 0.45, 0.475, 0.5, 0.525, 0.55, 0.575, 0.6, 0.625, 0.65, 0.675, 0.7, 0.725, 0.75, 0.775, 0.8, or 0.825, or between 0.025 and 0.1, or between 0.1 and 0.2, or between 0.15 and 0.4, or between 0.3 and 0.7, or between 0.5 and 0.85.

In an embodiment of the present invention, strontium calcium zinc silicate (Sr_{0.1}Ca_{1.9}ZnSi₂O₇) is obtained by combining Zn and Sr ions in the Ca—Si system by partly replacing Ca ions in Hardsytonite with Sr by a sol-gel method.

Other suitable biocompatible ceramic materials include biocompatible ceramic material comprising Baghdadite (Ca₃ZrSiO₉) disclosed in International PCT Publication No. WO 2009/052583, which is incorporated herein in its entirety. WO 2009/052583 describes an implantable medical device comprising biocompatible Baghdadite, in particular for regeneration or resurfacing of tissue.

Other suitable biocompatible materials also include a two phase or composite biocompatible ceramic material, wherein the first phase is a calcium zinc silicate and the second phase is a metal oxide, as disclosed in International PCT Publication No. WO 2012/162753, which is incorporated herein in its entirety. WO 2012/162753 describes a coating to improve the long-term stability of prior art implantable medical devices.

A further suitable biocompatible material is polycaprolactone-baghdadite (Ca₃ZrSi₂O₉) composite. Polycaprolactone (PCL) is a thermoplastic polymer that can be formed into fibres in a similar form to the UHMWPE fibres referred to herein. One suitable method to form PCL fibres is via electrospinning, although other methods will be apparent to the skilled person. PCL fibres can then be embedded with bioactive particles like baghdadite to enhance cell activity, and the hydrogel compositions disclosed herein. Preferably the PCL has high molecular weight to maximize strength. In one embodiment the molecular weight of PCL is around 90,000 g/mol, but in other embodiment could be higher, such as 120,000; 150,000; 200,000 or even 500,000 g/mol.

The biocompatible ceramic material, for example Sr—HT, is suitably present in the form of microparticles dispersed within the composition. Suitably the microparticles are dispersed evenly throughout the composition. Accordingly, the microparticles may be evenly dispersed around the plurality of fibres when in the scaffold. However, in other embodiments the microparticles are relatively concentrated at the exterior of the scaffold of the invention.

When in the form of microparticles, the biocompatible ceramic material (for example, Sr—HT) may have a diameter of between about 0.1 to about 500 μm, or between about 0.1 to 10 μm, or between 1 and 20 μm, or between 20 and 50 μm, or between 50 and 100 μm, or between 0.1 and 100 μm, or between 100 and 200 μm, or between 200 and 400 μm, or between 300 and 500 μm, or of less than 500 μm, or of less than 250 μm, or of less than 150 μm, for example, a diameter of 1, 25, 50, 75, 100, 125, 150, 175, 200, 225, 250, 275, 300, 325, 350, 375, 400, 425, 450, or 475 μm. The particle diameter may be an average particle diameter. In some embodiments, it is preferable to have a broad distribution of particle sizes, and in other embodiments it is preferable to have relatively narrow distribution of particle sizes.

The biocompatible ceramic material can be prepared in bulk and comminuted sufficiently to provide the required particle size, or synthetically prepared in the form of micro particles. Various synthesis methods will be known to the skilled person.

### Cell Adhesion Promoter

The synthetic implantable scaffold disclosed herein comprises a plurality of polymer fibres in contact with a composition, wherein the composition comprises a hydrogel-forming polymer and a biocompatible ceramic material. However, the composition may comprise one or more additional components. For example, in one embodiment, the composition herein comprises a hydrogel-forming polymer, a biocompatible ceramic material and a cell adhesion promoter.

Any suitable cell adhesion promoter may be used. For example, one suitable cell adhesion promoter is gelatin, which is a heterogeneous mixture of water-soluble proteins of high average molecular weights, present in collagen. The proteins are extracted by boiling skin, tendons, ligaments, bones, etc. in water. This can vary from manufacturer to manufacturer. Suitably the gelatin is derived from collagen, and in particular is an irreversibly hydrolyzed form of collagen. The addition of a relatively small quantity of gelatin promotes cell adhesion to the scaffold. Accordingly, in some embodiments, the composition contacted with or impregnated into the plurality of polymer fibres in the scaffold of the invention suitably also includes gelatin. However, other cell adhesion promoters will be known to those skilled in the art.

When used as a cell adhesion promoter in the present invention, gelatin is suitably not chemically crosslinked or chemically modified when incorporated into the hydrogel-forming polymer composition. Where the hydrogel-forming polymer is PVA, the gelatin is preferably not chemically crosslinked or chemically modified when incorporated into the PVA-based hydrogel, i.e., it is simply physically incorporated. Although other methods of preventing chemical modification of the gelatin will be known to those skilled in the art, the gelatin may be combined with the hydrogel-forming polymer (in some embodiments, PVA) and water and then heated and further mixed).

Suitably the concentration of cell adhesion promoter, for example gelatin, in the composition is between about 0.1 wt % and about 10 wt %. For example, the concentration may be between about 0.1 wt % and 0.5 wt %, or between 0.5 wt % and 5 wt %, or between 1 wt % and 4 wt %, or between 3 wt % and 7 wt %, or between 5 wt % and 10 wt %, or between about 0.5 wt % and about 2 wt %, e.g. between about 1 wt % and about 2 wt %, such as about 0.5 wt %, 1 wt %, 1.5 wt %, 2 wt %, 3 wt %, 4 wt %, 5 wt %, 6 wt %, 7 wt %, 8 wt %, 9 wt % or 10 wt %.

Suitable ratios of weight % (based on the weight of the composition) of hydrogel-forming polymer:gelatin are between 1:1 to 50:1, or between 1:1 and 10:1, or between 5:1 and 25:1, or between 20:1 and 40:1, or between 30:1 and 50:1, for example, 2, 3, 4, 5, 6, 7, 8, 9, 10, 15, 20, 30, or 40:1. More suitably, weight ratios of weight ratios of hydrogel-forming polymer:gelatin are between 5:1 to 15:1, or between 5:1 and 8:1, or between 7.5:1 and 12.5:1, or between 10:1 and 15:1, for example 6, 7, 8, 9, 10, 11, 12, 13, 14 or 15:1, particularly about 9:1.

Some suitable ratios of weight % (based on the weight of the composition) of hydrogel-forming polymer:gelatin:biocompatible ceramic material are: 9:1:4; or 10:1:5; or 5:1:2; or 15:1:10; or 5:1:10; or 15:1:2; or any ratios in between. About 9:1:4 is particularly suitable.

### Additional Components

Additionally or alternatively to a cell adhesion promoter, the scaffold of the invention may also include a bioactive glass, or a mixture of two or more bioactive glasses. Such materials usually contain Ca-phosphates and or Ca-sulphates. CaO, P₂O₅, SiO₂ and Na₂O are typical constituents for bio elements.

### Scaffold

As described herein, the composition of the invention is contacted with, or impregnated into, the plurality of polymer fibres. Where the composition is contacted with the plurality of polymer fibres, preferably each fibre in the plurality is in contact with the composition such that each fibre is at least partially covered by or encased in composition, or is completely (or substantially completely) covered by or encased in composition. However, depending on the fibre arrangement, only some of the fibres in the plurality may be in contact with the composition. For example, where the plurality of fibres is provided as a bundle (or as two or more bundles), outer fibres of the bundle(s) may be in contact with the composition, whereas inner fibres of the bundle(s) may not be in contact with the composition. This effect may be more pronounced as the diameter of the bundles increases. In some cases, where the plurality of fibres is provided as two or more bundles, some of the bundles may be completely (or substantially completely) covered by or encased in composition, and some of the bundles may be only partially covered by or encased in composition. The amount of contact between a fibre bundle and the composition may depend on where in the scaffold the bundle is located, e.g., at the surface or in the centre of the scaffold when looking at its cross-section, and/or on the final shape of the scaffold, e.g., a flat tape-like scaffold or a cylindrical scaffold.

The term ‘impregnated’ used herein is a form of contacting, most preferably one in which bundles of fibres are completely (or substantially completely) covered by or encased in composition and in which the composition permeates through or saturates the bundles. However, there may be circumstances where the bundles of fibres are impregnated with composition and the composition is not able to completely saturate or permeate through to each fibre in the bundles.

The contacting or impregnating as described herein may be conducted under pressure. Suitable methods of contacting or impregnating the plurality of fibres with composition to form scaffolds of the invention are described below in the section entitled ‘Method of preparing scaffold’.

Any suitable amount of composition may be used to contact with or impregnate the plurality of fibres in the scaffold according to the invention. Similarly, any suitable volume of fibres may be used in the scaffolds described herein. For example, in one embodiment, the fibre volume fraction of the scaffold is between about 5-95%. For example, the scaffold may comprise between 5 and 25% by volume polymer fibres, or between 10 and 30%, or between 25 and 50%, or between 40 and 60%, or between 50 and 75%, or between 60 and 90%, or between 70 and 95%, or between 50 and 95%, or about 5, 10, 15, 20, 30, 40, 50, 60, 70, 80, 90 or 95% by volume polymer fibres. In some embodiments, the impregnation composition fraction in the scaffold is within about 20-50 wt %, or within 20 and 30 wt %, or within 25 and 40 wt %, or within 30 and 45 wt %, or within 35 and 50 wt %, or within 30 and 50 wt %, or within 20 and 40 wt %, e.g., 20, 25, 30, 35, 40, 45, or 50 wt %.

It will be appreciated that there can be some porosity within the scaffold, which could be in the range of around 20 to 50 vol %, or within 20 and 30 vol %, or within 25 and 40 vol %, or within 30 and 45 vol %, or within 35 and 50 vol %, or within 30 and 50 vol %, or within 20 and 40 vol %, e.g., 20, 25, 30, 35, 40, 45, or 50 vol %.

The scaffold may have an equilibrium water content of between about 20 to about 80 wt %. For example, the scaffold may have an equilibrium water content of between about 20 to about 40 wt %, or between 30 to 50 wt %, or between 25 and 60 wt %, or between 40 and 80 wt %, or between 50 and 75 wt %, or between 60 and 70 wt %, or between 65 and 80 wt %, e.g., of 20, 25, 30, 35, 40, 45, 50, 55, 60, 65, 70, 75, or 80 wt %.

According to the invention, the cross-sectional shape of the synthetic implantable scaffold, when intended for use as a synthetic tendon or ligament, is similar to the natural tendon or ligament to be replaced, or is of a complementary shape to the natural tendon or ligament being repaired. Suitably the dimensions of the synthetic tendon or ligament scaffold are similar to the natural tendon or ligament to be replaced. However, more generally, the scaffold herein may take any suitable shape. For example, the scaffold may be in the form of a tape, ribbon or prismatic structure having any suitably shaped cross-section. For example, the cross-section may be circular, rectangular, square, trapezoidal, triangular, or have any other suitable cross-sectional shape. The scaffold may have a constant cross-sectional area along its length, or may have a cross-sectional area that varies with length, e.g., conical. The scaffold may take the form of a twisted prism or rope. In some embodiments, the scaffold may be cylindrical. In other embodiments, the scaffold may be a rectangular prismatic tape. In other embodiments, the scaffold may be an elongated rectangular or square prism. Any one or more of these scaffold shapes or profiles may be manufactured via techniques known to those of skill in the art, e.g., pultrusion, injection moulding, etc.

### Method of Preparing Composition

The present invention provides a method for preparing a composition comprising:
combining a hydrogel-forming polymer, a cell adhesion promoter, biocompatible ceramic microspheres, water and optionally acid (such as hydrochloric acid); and
mixing the components to achieve homogeneous mixture.

The method optionally further comprises adding a physical cross-linking agent to the hydrogel-forming polymer, cell adhesion promoter, biocompatible ceramic microspheres, water and optionally acid. In one embodiment, the physical cross-linking agent is a polyethylene glycol. In one embodiment, the physical cross-linking agent is PEG400. The physical cross-linking agent may be added in any suitable concentration, e.g., between about 1 and 50 wt %, or between 1 and 10 wt %, or between 10 and 20 wt %, or between 15 and 25 wt %, or between 25 and 35 wt %, or between 30 and 40 wt %, or between 35 q and 50 wt %, e.g., at 1, 5, 10, 15, 20, 25, 30, 35, 40, 45 or 50 wt % (where wt % is calculated as 100×[W_{agent}/(W_{agent}+Wₛₒₗᵤₜᵢₒₙ)] where Wₛₒₗᵤₜᵢₒₙ is the weight of the hydrogel-forming polymer solution).

For example, in one embodiment, the present invention provides a method for preparing a composition comprising:
combining PVA, gelatin, Sr—HT microspheres, water and acid; and
mixing the components to achieve homogeneous mixture.

In another embodiment, the present invention provides a method for preparing a composition comprising:
combining PVA, gelatin, Sr—HT microspheres, water, acid and PEG400; and
mixing the components to achieve homogeneous mixture.

In a further embodiment, the present invention provides a method for preparing a composition comprising the steps:
(a) combining PVA, gelatin, Sr—HT microspheres, and water;
(b) mixing the components to achieve a homogeneous mixture;
(c) adjusting the homogeneous mixture to a pH of between about 6.8 and 7.8;
(d) heating the homogeneous mixture to a temperature of between about 85 and 95° C.;
(e) adding PEG400 at a temperature of between about 85 and 95° C. dropwise to the mixture in step (d), with mixing.

In one embodiment, the PVA is in the form of a powder. In one embodiment, the gelatin is granulated.

Suitably the target pH range is about 7.0 to 7.5. Suitably the concentration of acid in the composition is such that a target pH of between about 7.0 to 7.5 is achieved. The target pH is suitably adjusted using acid, e.g., hydrochloric acid. Addition of acid suitably reduces the pH of the composition, which may have been raised by the alkalinity of the biocompatible ceramic microspheres.

Suitably the mixture is heated. For example, it may be heated to about 70 to 95° C., such as about 90° C. Heating the mixture assists dissolution.

The skilled person will appreciate that the viscosity depends on the percent concentrations of the components mixed. Viscosity of this gel during setting is also both temperature and time dependent.

### Method of Preparing Scaffold

The present invention provides a method for preparing a synthetic implantable scaffold comprising contacting a composition comprising a hydrogel-forming polymer and a biocompatible ceramic material with a plurality of polymer fibres. The present invention also provides a method for preparing a synthetic implantable scaffold, the method comprising the steps of:
providing a plurality of polymer fibres;
providing a composition comprising a hydrogel-forming polymer and a biocompatible ceramic material; and
contacting the plurality of polymer fibres with the composition to thereby form said synthetic implantable scaffold.

In some embodiments, the plurality of polymer fibres is provided in the form of a bundle. In other embodiments, the plurality of polymer fibres is provided in the form of two or more bundles.

In one preferred embodiment, the present invention provides a method for preparing a synthetic tendon or ligament scaffold, comprising the step of: impregnating an impregnation composition comprising a hydrogel-forming polymer and a biocompatible ceramic material into a bundle of polymer fibres.

In another embodiment, the present invention provides a method for preparing a synthetic implantable scaffold, the method comprising the steps of:
providing a plurality of polymer fibres;
providing a composition comprising a hydrogel-forming polymer, a biocompatible ceramic material and a cell adhesion promoter; and
contacting the plurality of polymer fibres with the composition to thereby form said synthetic implantable scaffold.

The method suitably further comprises the step of including gelatin in the composition. Suitably the hydrogel-forming polymer is PVA. Suitably the polymer fibres are UHMWPE fibres. Suitably the biocompatible ceramic material is Sr—HT.

Accordingly, in one embodiment, the present invention provides a method for preparing a synthetic implantable scaffold, the method comprising the steps of:
providing a plurality of UHMWPE polymer fibres;
providing a composition comprising PVA, a Sr—HT microparticles and gelatin; and
contacting the plurality of UHMWPE polymer fibres with the composition to thereby form said synthetic implantable scaffold.

In another embodiment, the present invention provides a method for preparing a synthetic implantable scaffold, the method comprising the steps of:
(a) combining PVA, gelatin, Sr—HT microspheres, and water;
(b) mixing the components to achieve a homogeneous mixture;
(c) adjusting the homogeneous mixture to a pH of between about 6.8 and 7.8;
(d) heating the homogeneous mixture to a temperature of between about 85 and 95° C.;
(e) adding PEG400 at a temperature of between about 85 and 95° C. dropwise to the mixture in step (d), with mixing;
(f) contacting the mixture formed in step (e) with a plurality of UHMWPE fibres;
(g) resting the product of step (f) at room temperature, thereby forming a hydrogel-based scaffold; and
(h) removing PEG400 from the hydrogel in step (g) through dialysis in water.

The composition contacted with the plurality of the polymer fibres may be made by a method according to the section entitled “Method of preparing composition” above.

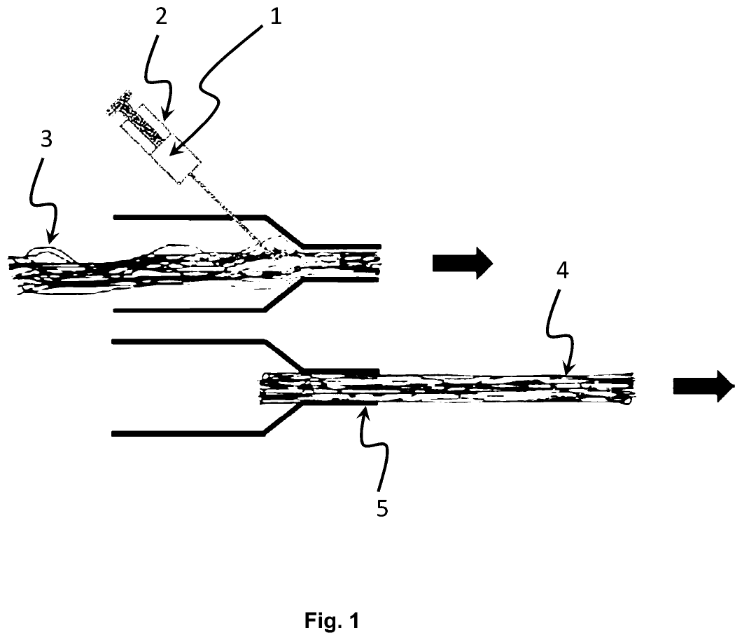

The skilled person will be aware of various methods to contact or impregnate the composition into a plurality of polymer fibres. One suitable method to contact or impregnate a bundle of polymer fibres is similar to a pultrusion process, whereby fibres are saturated with the composition in a barrel and then carefully formed and drawn through a narrow opening (die) which may be heated. Pultrusion results in straight constant cross-section parts of virtually any length. Such a method for preparing a synthetic tendon or ligament scaffold according to the invention is shown in FIG. 1, which is discussed below.

The composition may be combined with the fibres in the pultrusion barrel at elevated temperature. For example, the elevated temperature may be between 20 to 30, 30 to 40, 40 to 50, 20 to 50, 50 to 60, 60 to 70, 70 to 80, 50 to 80, 80 to 90, 85 to 95, 90 to 95, or 60 to 95° C. Alternatively, the composition may be combined with the fibres in the pultrusion barrel at room temperature and subsequently heated as the impregnated part is drawn through the die, which is at elevated temperature.

The diameter of the outlet through which the UHMWPE fibres and composition are drawn may be from about 2 mm to about 10 mm, such as from about 2 mm to about 6 mm, e.g. about 4 mm. The pultrusion speed can be any speed.

In some embodiments, a bundle of individual polymer fibres are pultruded, and in other embodiments individual polymer fibres are arranged into discrete primary bundles, and the primary bundles are pultruded. In yet other embodiments, secondary bundles (of primary bundles) are arranged and pultruded. It will be appreciated that the primary bundles of fibres (or secondary bundle of primary bundles, or tertiary bundles of secondary bundles, etc) are held together by the impregnation composition, which tends to bind and stick the fibres and bundles together to form a scaffold.

In some embodiments, the synthetic scaffold of the invention may be rested at room temperature (about 20° C.) for a time (e.g. about 5 minutes) after pultrusion.

In an alternative method of contacting or impregnating a plurality of polymer fibres, a bundle (or two or more bundles) of polymer fibres can be saturated with the composition by immersion, optionally with the application of pressure to force the composition into interstices within the fibres of the bundle(s) of fibres, and heating the composition to reduce the viscosity. Other methods will be known to the skilled person. The synthetic scaffold of the invention may be rested at room temperature (about 20° C.) for a time (e.g. about 5 minutes) after forming.

In some embodiments, the synthetic scaffold of the invention may be optionally immersed in deionized water (e.g. for 24 h), then freeze-dried for storage. The scaffold can then be rehydrated as necessary.

The present invention provides a synthetic tendon or ligament scaffold prepared by the method according to the invention.

### Medical Uses

The present invention is useful for partial or full tendon or ligament repair in a patient for ruptured or diseased tendon or ligaments. Accordingly, the present invention provides:
Use of the synthetic tendon or ligament scaffold of the invention for partial or full tendon or ligament repair.
A method of partial or full tendon or ligament repair in a patient comprising implantation of a synthetic tendon or ligament scaffold of the invention
A synthetic tendon or ligament scaffold of the invention for use in partial or full tendon or ligament repair in a patient.
Use of a synthetic tendon or ligament scaffold of the invention in the manufacture of a medicament for partial or full tendon or ligament repair in a patient.

In addition, the present invention provides:
An impregnation composition comprising: a hydrogel-forming polymer (such as PVA); cell adhesion promoter (e.g. gelatin); and Sr—HT; for use in partial or full tendon or ligament repair in combination with a bundle of UHMWPE fibres.
Use of an impregnation composition comprising: a hydrogel-forming polymer (such as PVA); gelatin; and Sr—HT in the manufacture of a synthetic tendon or ligament scaffold.
Use of an impregnation composition comprising: a hydrogel-forming polymer (such as PVA); gelatin; and Sr—HT in the manufacture of synthetic tendon or ligament scaffold for partial or full tendon or ligament repair in combination with a bundle of UHMWPE fibres.

The present invention further provides:
A synthetic tendon or ligament comprising: a plurality of synthetic tendon or ligament scaffolds according to the invention. In one embodiment the synthetic tendon or ligament is in the form of an aponeurosis.
Use of a plurality of the synthetic tendon or ligament scaffolds in the manufacture of a prosthesis for partial or full tendon or ligament repair.
A prosthesis comprising a plurality of the synthetic tendon or ligament scaffolds for partial or full tendon or ligament repair.

The scaffold of the invention is advantageously an artificial ligament used for repairing or replacing any ligament in animals, in particular non-human mammals or humans. Ligaments which may be repaired or replaced may be selected from the following: head and neck ligaments (cricothyroid ligament, periodontal ligament, suspensory ligament of the lens), wrist ligaments (palmar radiocarpal ligament, dorsal radiocarpal ligament, ulnar collateral ligament, radial collateral ligament), shoulder ligament (rotator cuff), knee ligament (anterior cruciate ligament (ACL), lateral collateral ligament (LCL), posterior cruciate ligament (PCL), medial collateral ligament (MCL), cranial cruciate ligament (CrCL)—quadruped equivalent of ACL, caudal cruciate ligament (CaCL), patellar ligament). In one embodiment the patient is a human.

It will be appreciated that in relevant embodiments, both the PVA hydrogel and UHMWPE components are essentially non-degradable in normal physiological environments, and would remain substantially in vivo during the lifetime of a prospective patient.

There are a variety of anchors used to fix the ends of a ligament scaffold into bone. Most commonly, they are so-called interference screws, designed to be inserted along the scaffold (prosthesis) (transplanted tendon or ligament, or an artificial ligament) within an anchor hole, or tunnel, drilled in the bone. The interference screw jams the prosthetic tissue against the bone within the anchor hole. Such screws are made either from metal, most commonly titanium, or bioresorbable polymers. Another common technique is so-called cross-pin used to anchor a loop of the prosthetic tissue within a hole drilled in the femoral condyle. In all cases, prosthetic tissue exits the tunnel by bending over the edge of the bone; healing/remodeling of the bone is expected to fill the gaps and to result in a natural-like anchorage of the ligament in the bone. The ends of a synthetic implantable scaffold according to the invention can be attached or tied to an anchor point (e.g., another scaffold, such as a porous cancellous bone scaffold) to create a synthetic bone-tendon-bone complex. Methods of anchoring are well known to the skilled person and all suitable methods fall within the purview of the present invention.

### Advantages of the Invention

The scaffold of the invention provides one or more advantageous properties, especially over biological-based replacements, for example:
a method of production which provided controlled and predetermined scaffold size and diameter
long-term off-the-shelf storage;
batch-to-batch consistency;
high mechanical strength;
high toe-linear modulus;
high equilibrium water content; and
enhanced cell proliferation properties.

Further, use of the hydrogel in the present invention is able to retain its overall water content as water diffuses in and out of the scaffold in vivo, effectively meaning that the hydrogel acts as a long-term lubricant. The hydrogel remains in vivo during the lifetime of a prospective patient. This is advantageous over existing technology employing a lubricating agent wherein the lubricating agent is a fluid which may eventually diffuse out of the initial implant, and not act as a lubricant in the long-term, potentially resulting in synovitis.

One of the most remarkable findings by the inventors was that, surprisingly, it was possible to significantly increase the tensile strength and modulus of UHMWPE uniaxial fibre scaffolds by approximately 40%, by impregnating the fibres with a PVA hydrogel which by itself is orders of magnitude weaker than UHMWPE, and has little or no significant tensile strength. The tensile mechanical values of PVA-UHMWPE, PG-UHMWPE and PSG-UHMWPE lie within range of those reported in literature for human Achilles' tendon tissues, and exceed those of the values for anterior cruciate ligaments and many other synthetically developed and decellularized biological tendon grafts. Surprisingly, two distinct tensile moduli for the synthetic tendon scaffolds of the invention have been observed prior to the yield strain, similar to those observed in the native tendon. The hierarchical nature of biological tissues such as bone and tendons often result in mechanical properties that surpass the theoretical value based on the volume fraction of its component parts, and may explain the extraordinary tensile properties of tendons and ligaments despite a high water content. Without wishing to be bound by theory, the inventors propose that the increase in the overall tensile strength and modulus may be due to the following factors: firstly due to the covering of defects on the surface of UHMWPE fibres, similar to how polymer coatings improve the mechanical properties of brittle materials under tension; secondly due to a more even distribution of applied tensile load through the impregnating hydrogel; and thirdly due to the individual fibres being able to glide past relative to one another with minimal friction. To achieve this, it is preferable that the hydrogel itself cover substantially all of the fibre bundles, and also should be able to withstand high local compressive, tensile and shear strains without failure or plastic deformation, which was made possible by using injectable PVA, PG and PSG hydrogels. The individual effect of gelatin and SrHT particles on the physical properties on the overall scaffold however appear negligible due to the magnitude of the forces applied.

### EXAMPLES

### Specific Embodiment

In one embodiment, the present invention provides a novel synthetic tendon or ligament scaffold consisting of longitudinally aligned ultra-high molecular weight (UHMWPE) fibres capable of uniaxial load-bearing, which have been impregnated with a polyvinyl alcohol (PVA)-based hydrogel to mimic the fibre-ECM hierarchical structure of native tendons or ligaments. Suitably, the impregnation composition (hydrogel composition) consists of multiple components: PVA, gelatin and Sr—Hardystonite (Sr-HT). PVA provides the necessary porous architecture for retention of water molecules; gelatin allows for cell adhesion; and Sr—HT allows for improved cell activity. This new synthetic scaffold shows simultaneous high mechanical strength, high toe-linear modulus and high equilibrium water content similar to native tendon, as well as enhanced in vitro mesenchymal stem cell proliferation properties.

### Synthesis of Particles of Biocompatible Ceramic Material

Sr—HT ceramic micropowders were prepared by the sol-gel process using tetraethyl orthosilicate ((C₂H₅O)₄Si, TEOS), zinc nitrate hexahydrate (Zn(NO₃)₂.6H₂O), calcium nitrate tetrahydrate (Ca(NO₃)₂.4H₂O) and strontium nitrate (Sr(NO₃)₂) as raw materials (all from Sigma Aldrich, USA). The TEOS was mixed with water and 2 M HNO₃ (mol ratio: TEOS/H₂O/HNO₃=1:8:0.16) and hydrolyzed for 30 min under stirring. Then, the Zn(NO₃)₂.6H₂O, Ca(NO₃)₂.4H₂O and Sr(NO₃)₂ (5 wt. %) solutions were added into the mixture (mol ratio: TEOS/Zn(NO₃)₂.6H₂O/Ca(NO₃)₂.4H₂O=2:1:2), and reactants were stirred for 5 h at room temperature. After the reaction, the solution was maintained at 60° C. for 1 day and dried at 120° C. for 2 days to obtain the dry gel. The dry gel was calcined at 1200° C. for 3 h. The calcined powder was then subsequently grinded in ethanol using a planetary ball mill (Retsch, UK) for 3 h at 150 rpm and sieved through 25 micrometer mesh.

Sr—HT microparticles in the size range of about 1-10 micrometers are prepared by grinding in planetary ball mill. Using 150 revs per min for about 3 h, the final particle average size was about 1.5 micrometers. Of course the skilled person will appreciate that this can be varied by changing the rev per min and time grinding.

### Synthesis of Compositions

Polyvinyl alcohol (PVA) with Mw=89,000-98,000 (Sigma Aldrich, USA) and granulated bovine gelatin (Government Department Stores, Australia) were commercially obtained.

Four different PVA hydrogel groups were synthesized. Firstly, PVA15% (PVA), PVA13.5%-Gelatin1.5% (PG), PVA15%-SrHT (PS) and PVA13.5%-SrHT-Gelatin1.5% (PSG) solutions were prepared, with its constituents as outlined in Table 1:

TABLE 1
Composition of solutions prior to formation of hydrogel

| | PVA | Gelatin | SrHT |
|---|---|---|---|
| wt % calculation | $\frac{{w}_{PVA}}{{w}_{PVA + Gelatin + water + HCl}}$ | $\frac{{w}_{gelatin}}{{w}_{PVA + Gelatin + water + HCl}}$ | $\frac{{w}_{SrHT}}{{w}_{PVA + Gelatin + water + HCl}}$ |
| PVA | 15% | 0% | 0% |
| PVA-Gelatin1.5% | 13.5% | 1.5% | 0% |
| (PG) | | | |
| PVA-SrHT (PS) | 15% | 0% | 6% |
| PVA- | 13.5% | 1.5% | 6% |
| SrHTGelatin1.5% | | | |
| (PSG) | | | |

The total macromer concentration was made up to 15 wt % for the hydrogels. For the PS and PSG solutions, 3.3 mL of 1M hydrochloric acid per 1.0 g Sr—HT was added to the solution to neutralize the alkaline effect of SrHT powder in PVA solution alone (pH about 9.2 without the acid). The target pH was about 7.0 to 7.5. The constituents for the solutions were dissolved and mixed thoroughly at 90° C.

For the synthesis of the hydrogels, PEG400 (Sigma Aldrich, USA) and PVA, PG, PS and PSG solutions were first heated in a microwave for the solutions to reach about 90° C. The heated PEG400 was then added dropwise into the PVA, PG, PS and PSG solutions whilst simultaneously subjecting the mixture under vortex mixing to initiate gelation. Care was taken as rapid incorporation of PEG400 resulted in irreversible localized crystallization of PVA. The amount of PEG400 added to the solution was 28 wt %, where the weight percent was calculated as follows: 100×(W_{PEG}/(W_{PEG}+Wₛₒₗᵤₜᵢₒₙ)), where Wₛₒₗᵤₜᵢₒₙ was the weight of the PVA solution used. After the vortex mixing, the hot hydrogel solution was then transferred to 50 mL falcon tubes and cooled to room temperature for gelation completion and storage. During the cooling process, the flask was sealed to minimize the temperature gradient, and to continuously agitate the hydrogel to prevent a “skin layer” at the hydrogel/air interface from forming.

Use of PEG400 has a beneficial effect, in that it assists in inducing formation of the hydrogel—without adding PEG400 the PVA solution tends to remain as a liquid solution (see U.S. Pat. No. 7,776,352). Whilst the PEG400 is part of the synthesis procedure, it is removed after gelation through dialysis in water, thereby replacing the PEG400 with water inside the pores of the hydrogel component of the scaffold. The scaffold can then be freeze dried, leaving little or no liquid component inside the scaffold in its dried state.

### Polymer Fibres

Ultra-high molecular weight polyethylene (UHMWPE) fibres were obtained commercially and used as delivered (Goodfellow, UK). Fibres were obtained under the trade name Dyneema, in the form of ‘multifibre yarns’, tex number=145(±10%), and number of fibres=1300(±10%). Individual yarns were manually isolated, cut to required length and then a number of yarns were regrouped to form uniaxial fibrous scaffolds to the required diameter—20 yarns for 2 mm diameter samples, 80 yarns for 4 mm diameter samples. The skilled person will appreciate that when a plurality of fibres are twisted it is common in the art to refer to the resulting twisted fibre bundle as a yarn.

### Method of Preparing the Scaffold

Referring to FIG. 1, a method of production of the synthetic tendon and ligament scaffold of the invention is shown.

The homogeneous mixture of impregnation composition is heated at about 90° C. for a period of 2 to 20 minutes. The heated mixture 1 is then transferred to a syringe 2 and the heated mixture 1, which is in the form of a gel, is injected into the internal core of UHMWPE fibre bundle 3. The rheology of the impregnated tendon or ligament scaffold 4 is determined by the mixture of the components during the preparation of the hydrogel. The impregnated tendon or ligament scaffold 4 is then pultruded through an opening 5 of predetermined geometry and size, for example a 4 mm diameter outlet. The pultrusion rate may be about 5 mm/s to 10 mm/s.

The impregnated tendon or ligament scaffold 4 can then be ‘rested’ for about 5 minutes at room temperature (about 20° C.), followed by immersion in deionized water for 24 h, and then freeze-drying for storage. The scaffold can then be rehydrated and used as necessary.

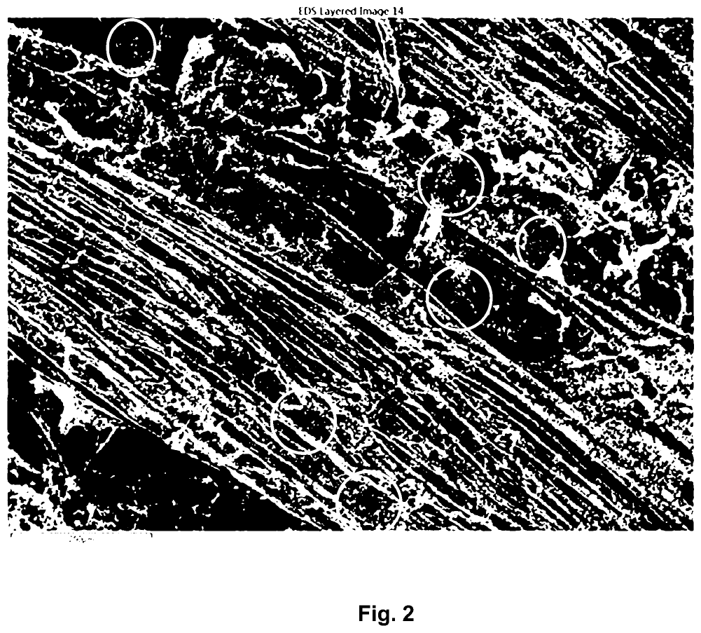

FIG. 2 is a SEM of a freeze-dried tendon or ligament scaffold according to the invention. The UHMWPE fibres 6 can clearly be seen, and close inspection of the figures shows that the fibres are coated with PVA/gelatin hydrogel. The Sr—HT microparticles can also be seen evenly dispersed throughout the fibre bundle 3 (e.g. several particles have been highlighted in the FIG. 2).

### Tensile Mechanical Properties

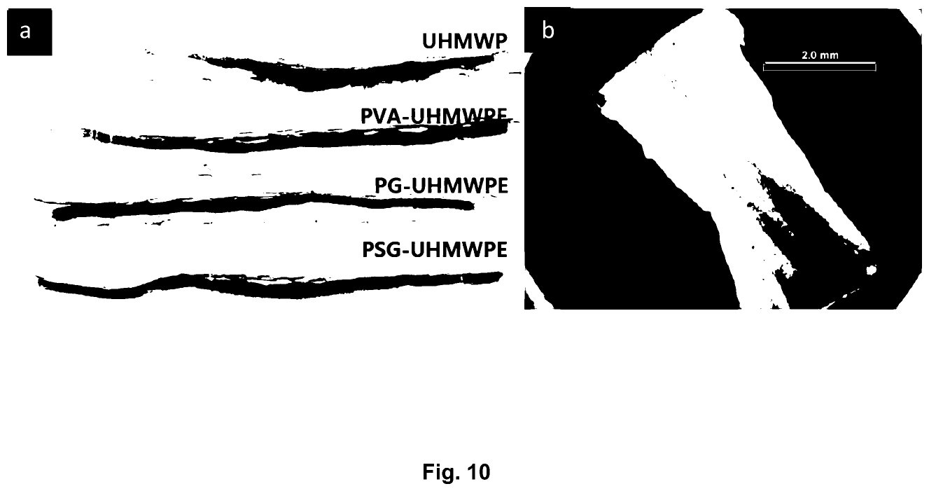

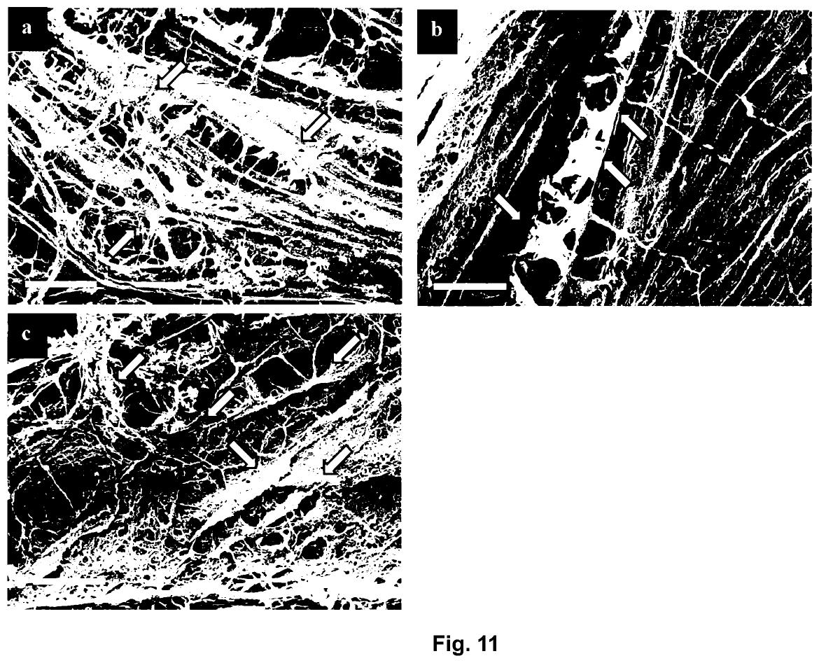

Cylindrical specimens with a testing region of 40 mm in length and 2 mm in diameter (20 yarns per specimen) were prepared. UHMWPE and lyophilized PVA-UHMWPE, PG-UHMWPE and PSG-UHMWPE scaffolds were immersed in 1× phosphate buffered saline (PBS) pH 7.2 at 37° C. for 24 hours to fully hydrate the samples. See FIG. 10a for photographs of the 4 samples prepared. The samples were then tested for their tensile strength and modulus using 1 kN load cell, cross-head speed of 10 mm/min, with ends clamped with pneumatic grips at 500 kPa. The tensile yield strength was measured as the highest stress value shown in the stress-strain curve obtained at the end of the elastic region, with the yield strain obtained as the strain at the tensile yield strength. The tensile modulus of the toe region at tensile stress=5 MPa, and the linear elastic region at tensile stress=30 MPa, were separately obtained by using linear regression. Three samples for each material were examined.

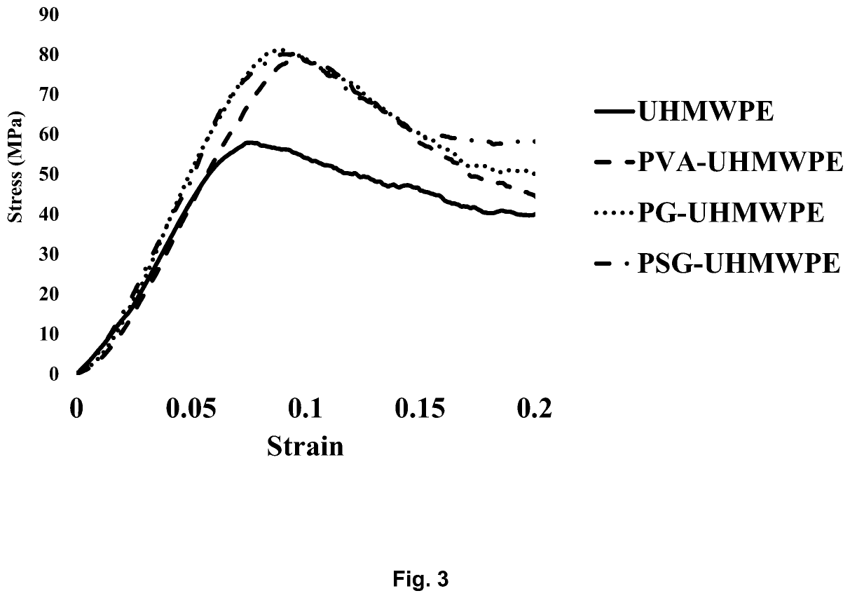

The FIG. 3 graph shows the representative tensile stress strain curves of each scaffold group, with the average and standard deviation of tensile yield strength, yield strain, and moduli at stress=5 MPa and stress=30 MPa recorded in Table 2. PVA-UHMWPE, PG-UHMWPE and PSG-UHMWPE all show significantly higher tensile yield strength and yield strain compared to unmodified wet UHMWPE fibres, with approximately 40% increase in tensile yield strength. Tensile modulus remained similar between all groups at both tensile stresses of 5 MPa and 30 MPa (Table 2). No samples showed failure of the test specimen at tensile strains up to 20%, though individual fibres within the scaffolds appeared to first fail at the clamped regions of the test specimen.

Table 2 provides tensile mechanical properties expressed as mean±standard deviation for UHMWPE, PVA-UHMWPE, PG-UHMWPE and PSG-UHMWPE tested. Experimental values are compared to the values for the human Achilles' tendon reported in the prior art.

TABLE 2
Tensile mechanical properties expressed as mean ± standard deviation

| | | | | Tensile |
|---|---|---|---|---|
| | Yield | | Tensile | modulus at |
| | strength | Yield strain | modulus at | 30 MPa |
| Specimen | (MPa) | (%) | 5 MPa (toe) | (linear) |
| Wet | 56.3 ± 3.0 | 6.9 ± 0.6 | 727 ± 59 | 1100 ± 160 |
| UHMWPE | | | | |
| PVA- | 77.2 ± 7.7* | 9.5 ± 0.4* | 658 ± 27 | 1123 ± 168 |
| UHMWPE | | | | |
| PG-UHMWPE | 77.0 ± 5.0* | 9.9 ± 1.3* | 638 ± 64 | 1245 ± 130 |
| PSG- | 81.8 ± 2.4* | 9.0 ± 1.0* | 712 ± 140 | 1279 ± 103 |
| UHMWPE | | | | |
| Human | 79 ± 22 | 8.8 ± 1.9^{&} | — | 819 ± 208^{#} |
| Achilles' | | | | |
| tendon | | | | |

*p < 0.05 compared to wet UHWMPE only
^{&}failure strain
^{#}modulus of linear region

All data is presented as mean±SD. For statistical analysis, Levene's test was performed to determine the homogeneity of variance of data, and then either Tukey's HSD or Tamhane's post hoc tests were used. SPSS software (IBM) was used for all statistical analyses and differences were considered as significant if p<0.05.

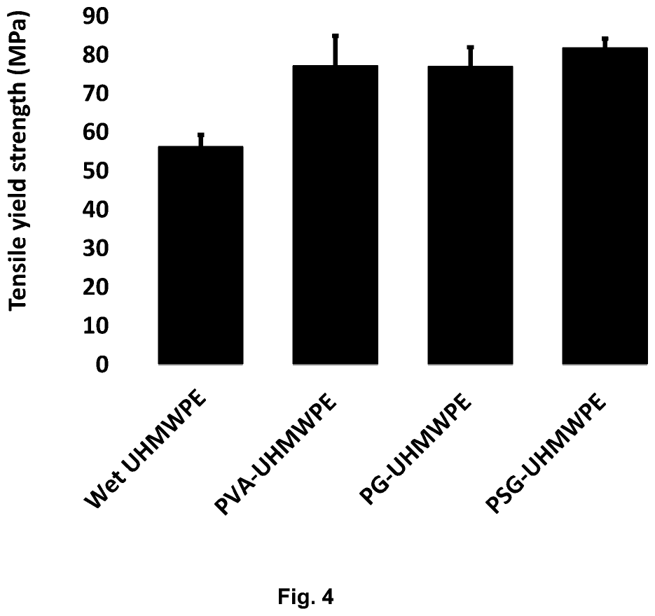

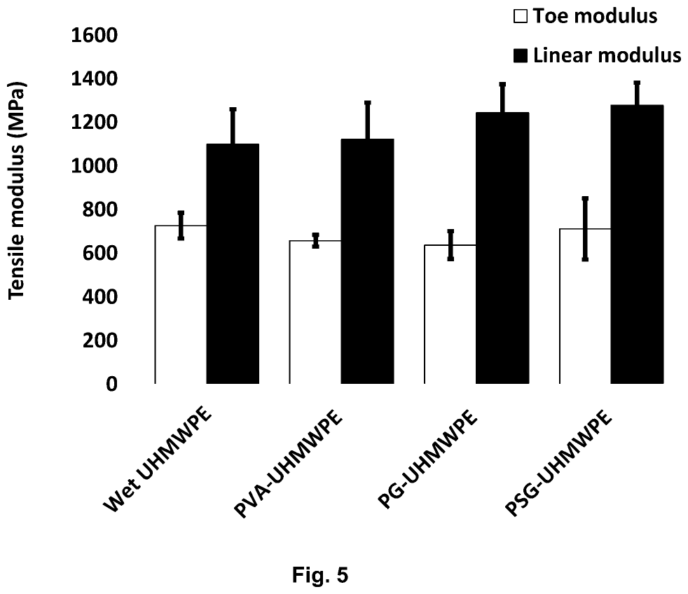

A synthetic tendon or ligament scaffold made according to the invention demonstrated high tensile strength (about 57 MPa) and modulus (about 500 MPa at toe region, about 800 MPa at linear region), exceeding those reported for rotator cuff tendons and anterior cruciate ligaments, and within range for Achilles' tendon (see FIG. 4 and FIG. 5). From FIG. 4, it can be seen that the tensile strength increases slightly when the PVA and gelatin are added. However, FIG. 5 shows that the addition of gelatin provided a surprising improvement in tensile modulus compared to the fibre/PVA scaffold, with the modulus increasing from about 400 to 800 MPa (linear region). The toe region also experiences an increase in modulus.

### Equilibrium Water Content

Dry UHWPE and lyophilized PVA-UHMWPE, PG-UHMWPE and PSG-UHMWPE scaffolds were first weighed on an electronic scale for their initial dry mass (w_{dry}). The samples were then immersed in 1× phosphate buffered saline (PBS) pH 7.2 at 37° C. for 2 hrs. The samples were then removed from the PBS and carefully blot-dried using clean paper towels to remove excess moisture. The swollen sample weight was then measured (w_{swollen}). The equilibrium water contents of the samples were calculated using the following formula: 100×(w_{swollen}−w_{dry})/w_{swollen}. Four samples for each material were examined.

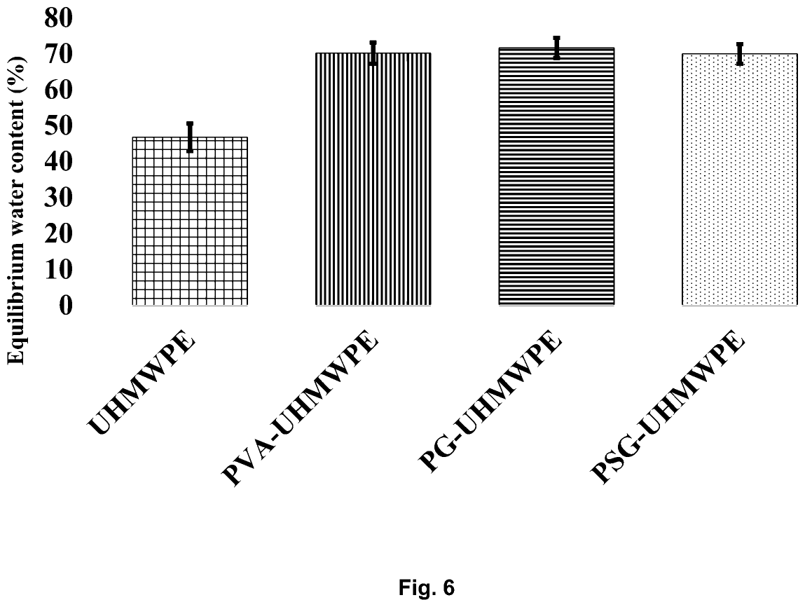

The synthetic tendon or ligament scaffold made according to the invention displayed high equilibrium water content, which is similar to that of a native tendon or ligament, e.g. about 70 wt % (see FIG. 6). As can be seen from FIG. 6, UHMWPE scaffolds showed 47±4% equilibrium water content. Scaffolds made from the longitudinal UHMWPE fibres alone had to be tied at either end, and had limited capacity to retain water content, through the physical entrapment of water molecules rather than absorption. In contrast, the PVA-UHMWPE, PG-UHMWPE and PSG-UHMWPE had significantly higher equilibrium water content of 70±3%, 72±3% and 70±3% respectively compared to UHMWPE. No significant difference was observed between the PVA-UHMWPE, PG-UHMWPE and PSG-UHMWPE groups.

### Ovine Mesenchymal Stem Cell Attachment and Proliferation

PVA-UHMWPE, PG-UHMWPE and PSG-UHMWPE scaffolds of size 6×5×1 mm were prepared for ovine mesenchymal stem cell proliferation study, by extruding the PVA, PG or PSG hydrogel injected into 40 UHMWPE yarns through a 4 mm diameter channel and manually flattening the sample directly after extrusion. Heterologous oMSCs were isolated from the iliac crest of Merino sheep by Ficoll separation and differential adhesion. All oMSCs used in the experiments were at passage 9. For oMSC attachment (n=2) and proliferation (n=4) studies, 1.0×10⁴ cells were seeded per sample. The cells were cultured in complete medium containing α-minimal essential medium (α-MEM) (Gibco Laboratories, USA), supplemented with 10% (v/v) heat-inactivated fetal calf serum (FCS) (Gibco Laboratories, USA), and 100 U ml⁻¹ penicillin+100 micrograms ml⁻¹ streptomycin (Gibco Laboratories, USA). The cells were incubated in 37° C. with 5% CO₂, and complete medium changes were performed every 3 days.

For oMSC attachment morphology, the cells were seeded and cultured for 24 h before SEM observation. Prior to SEM imaging, the samples were fixed in 4% paraformaldehyde solution for 30 mins, then rinsed in PBS several times. The samples were then frozen at −80° C., lyophilized and sputter coated with gold under vacuum. To evaluate oMSC proliferation, the CellTiter 96 Aqueous Assay (Promega, USA) was used to determine the number of viable cells on the cultured scaffolds via a colorimetric method. The assay solution is a combination of tetrazolium compound (3-(4,5-dimethylthiazol-2-yl)-5-(3-carboxymethoxyphenyl)-2-(4-sulfophenyl-2H-tetrazolium), MTS) with an electron coupling reagent (phenazine methosulfate) at a volume ratio of 20:1. The former compound can be bioreduced by viable cells into formazan, which is soluble in cell culture medium, and the absorbance of formazan at 490 nm is directly proportional to the number of viable cells present. oMSC proliferation was evaluated after 3 and 7 days of culture. At each time point, the culture medium was replaced by 200 microlitres of the MTS working solution, which consisted of the CellTiter 96Aqueous Assay solution diluted in PBS at a volume ratio of 1:5. After 4 h of incubation at 37° C., 100 microlitres of the working solution was transferred to a 96-well cell culture plate, and the absorbance at 490 nm was recorded using a microplate reader (PathTech, Australia) using the software Accent.

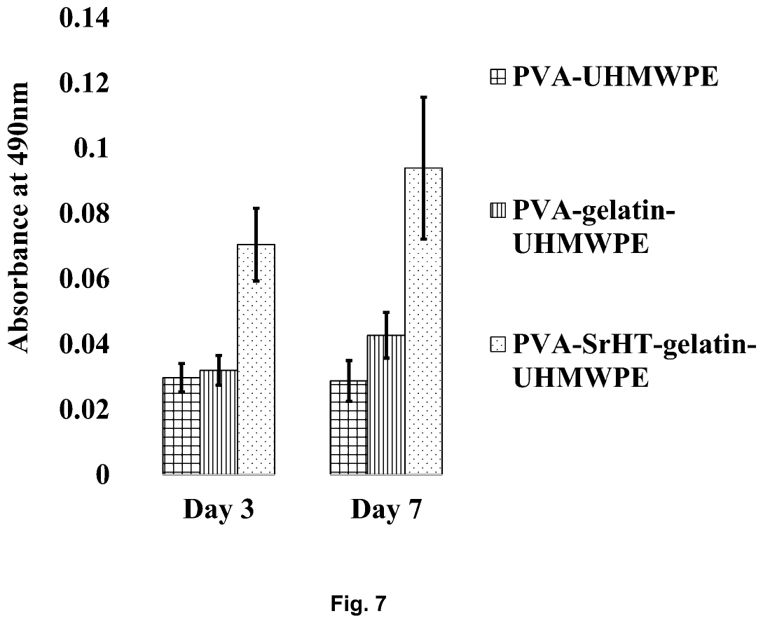

The synthetic tendon or ligament scaffold made according to the invention showed enhanced in vitro mesenchymal stem cell proliferation properties. More specifically, FIG. 7 shows increased ovine mesenchymal stem cell proliferation of UHMWPE/multicomponent hydrogel vs UHMWPE/PVA hydrogel and UHMWPE/PVA-gelatin hydrogel. The cell proliferation assay showed that the absorbance values for the PSG-UHMWPE was significantly higher than PVA-UHMWPE and PG-UHMWPE at both day 3 and 7 of oMSC culture. There was no significant difference between PVA-UHMWPE and PG-UHMWPE for both day 3 and day 7 time points.

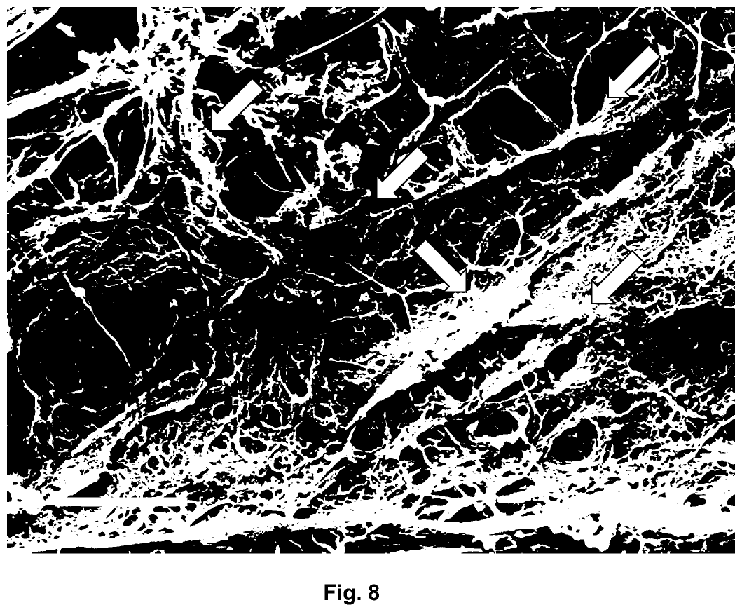

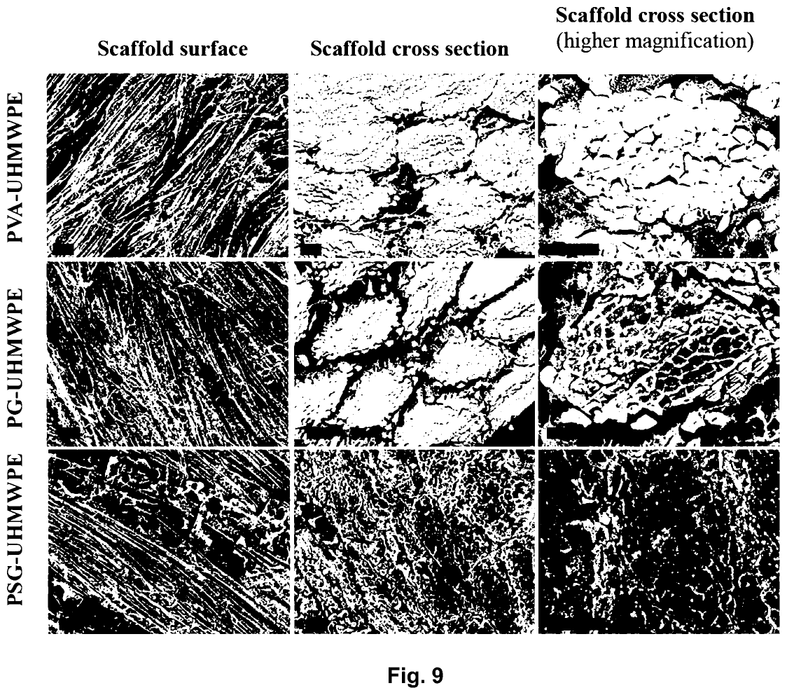

FIG. 8 shows the scanning electron microscopy images, with arrows pointing to what appear to be oMSCs with flattened, fibroblastic morphology. Cellular processes could also be observed, with most of these processes running transverse to the fibre direction for all three groups examined.

In order to mimic the extracellular ground substance (a gel-like component of the various connective tissues), the inventors were able to penetrate the PVA-hydrogel into the internal core of the UHMWPE scaffold and surround the fibre bundles using an injectable form of PVA-hydrogel—this resulted in an wholly intact fibrous scaffold where all the fibres were in the longitudinal direction without the need for horizontally woven fibres, nor stress inducing knots at either end to hold the fibrous structure together (FIG. 8).

All hydrogel-fibre scaffolds tested were shown to be biocompatible and supporting the attachment of oMSCs after 24 h of culture.

In terms of cell viability, the inventors were able to show improved ovine mesenchymal stem cell proliferation on PSG-UHMWPE compared to both PVA-UHMWPE and PG-UHMWPE. The incorporation of gelatin alone in the theta-gelled PVA hydrogel appeared to have an insignificant effect on the cell proliferation on these scaffolds, and the presence of Sr—HT in the hydrogel structure enhanced cell proliferation. The presence of the underlying aligned UHMWPE did not appear to influence negatively on the oMSC proliferation.

### In Vivo Study

### SUMMARY

To evaluate the biomechanical and biological characteristics of a scaffold of the invention as a tendon replacement, a scaffold (PSG-UHMWPE) was implanted into the right Achilles tendon of nine New Zealand White (NZW) rabbits after creating a 5 mm tendon defect. The PSG-UHMWPE scaffold used was 5 mm in diameter and 10 mm in length.

An additional group of nine NZW rabbits served as control. In this group, a tenotomy of the right Achilles tendon was performed and immediately treated by a primary tendon suture. Clinically, tendon sutures represent the gold standard in the treatment of tendon injury.

After three months of healing time, all animals were euthanized and macroscopic and biomechanical examination of the Achilles tendon was conducted.

To evaluate the initial strength of tendons treated either by primary tendon suture or by scaffold implantation, the left (non-operated) Achilles tendons were harvested after sacrifice and tendon suturing (n=5) or tendon grafting (n=5) was performed. These samples were immediately subjected to mechanical testing. Furthermore, the mechanical properties of native tendon tissue of non-operated (left) hind limbs were determined (n=6). Non-implanted samples of the scaffolds (length: 25 cm) were investigated biomechanically and histologically.

### Materials and Methods

### Animal Model and Surgical Procedure

18 female New Zealand White rabbits at the age of 4 months (mean body weight (BW): 2.89 kg±0.29 kg) were randomly divided into two intervention groups of equal numbers: implantation of the scaffold and primary tendon suture.

The animals were anesthetized by intravenous application of ketamin (7.5-15 mg/kg BW) and xylazin (0.5-1.0 mg/kg BW) and their right hind limb was shaved and prepared for sterile operative intervention.

Before implantation, all scaffolds were hydrated in sterile saline for 2 hours.

Surgical access to the Achilles tendon was achieved by a lateral skin incision of about 2-3 cm length. Afterwards the crural fascia and the paratendon were incised and the Achilles tendon was separated from the tendon of the M. flexor digitalis superficialis.

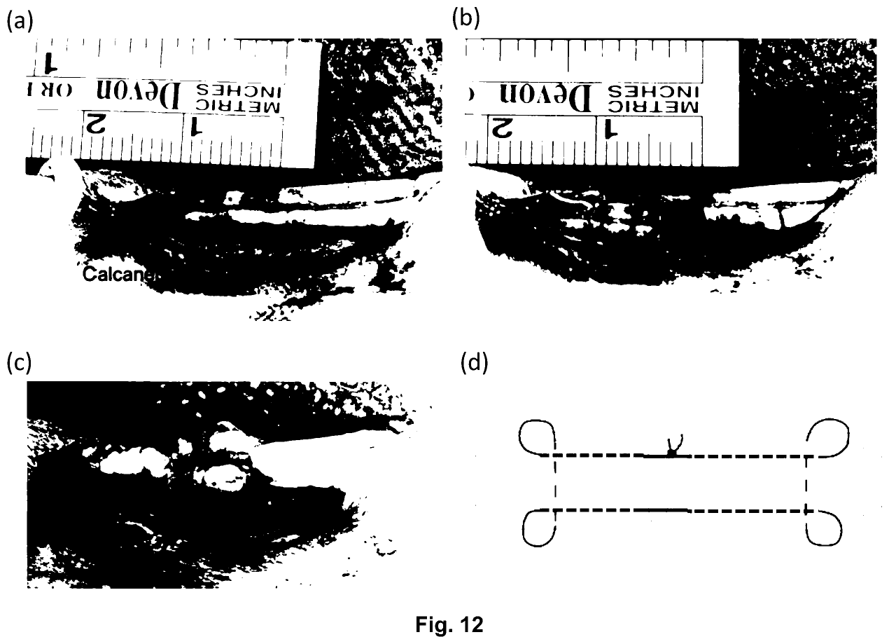

In the control group, a tenotomy (2 cm proximal of the calcaneus) was performed (FIGS. 12 (a)-(b)) and the tendon stumps were re-adapted using a Kirchmayr-Kessler tendon suture (suture material: PDS 4-0) (FIGS. 12(c)-(d)).

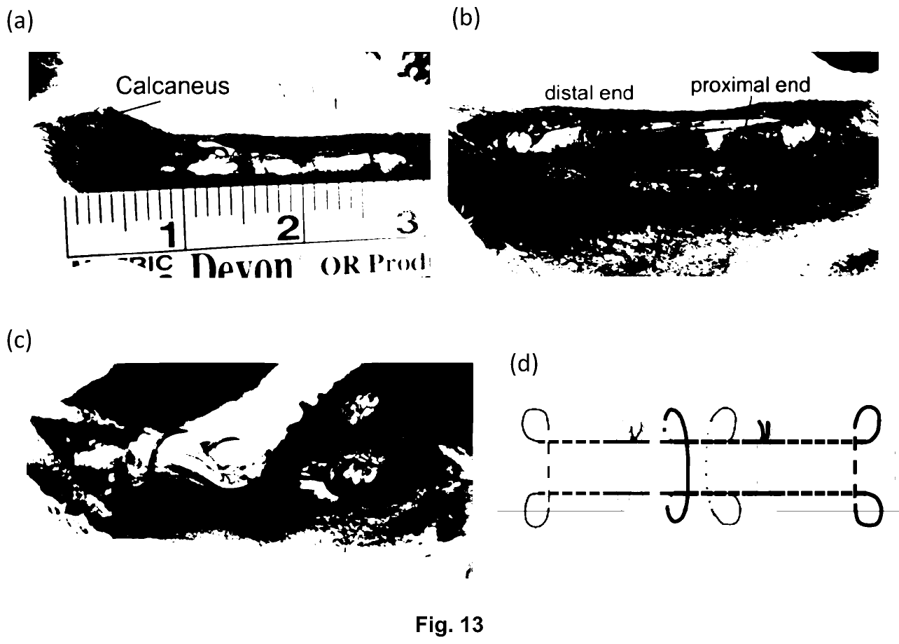

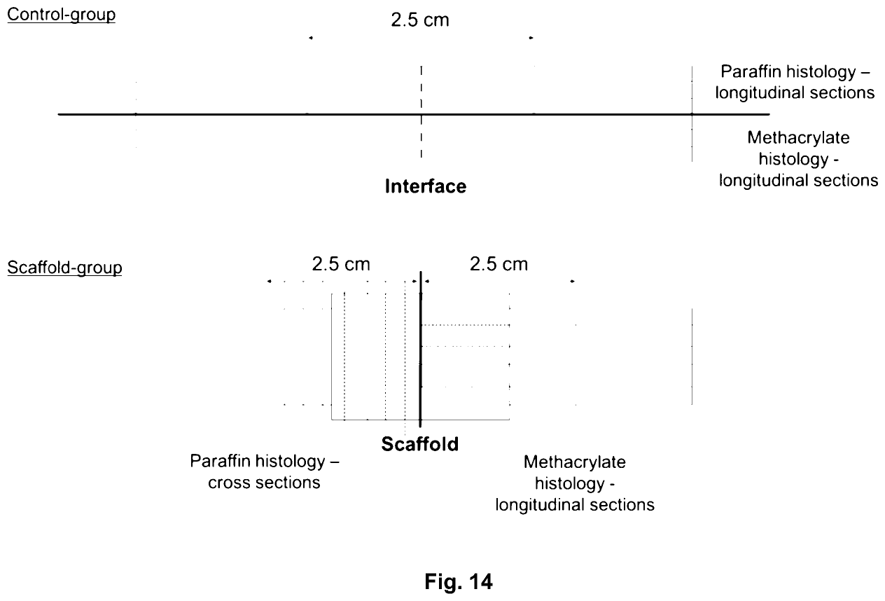

For the implantation of the scaffold, a 5 mm tendon defect was created in the right Achilles tendon (1.5 cm-2 cm proximal of the calcaneus; FIGS. 13(a)-(b)). The tendon stumps were sutured to the scaffold by a modified Kirchmayr-Kessler-suture (FIGS. 13(c)-(d)). By this modification, multiple needle penetration of the scaffold was avoided and thus destruction of the fiber arrangement of the scaffold was prevented.

### Macroscopic Examination

Directly after sacrifice, both hind limbs of each rabbit were dissected and macroscopically evaluated. Thereby the scoring system by Stoll et al. Healing parameters in a rabbit partial tendon defect following tenocyte/biomaterial implantation. Biomaterials (2011) 32: 4806-4815 (incorporated herein by reference) was applied and a grade between 0 and 17 was assigned to each sample (completely intact tendon: grade 17).

### Biomechanical Examination

All tests were performed at room temperature (20-22°) using a standard materials testing machine (Zwick GmbH und Co.KG, Ulm, Germany). Measurements and data acquisition were carried out using TestXpert II (Zwick GmbH und Co.KG, Ulm, Germany). During testing, specimens were cyclically preconditioned (5 cycles) between 5 N and 40 N at a constant velocity of 60 mm per minute. Afterwards a tensile test to failure was conducted. For each sample diameter and length of the specimen as well as applied force, deformation and time were recorded during testing.

Maximum force (Fₘₐₓ (in N)), maximum stress (σₘₐₓ (in %)), and maximum strain (εₘₐₓ (in MPa)), were automatically determined by the testing software. Young's modulus (E in Mpa) and stiffness (κ (in N/mm)) were calculated using a custom-made MATLAB program (The MathWorks®, Inc., USA).

$$Stress ( σ ) in MPa \text{:} σ = \frac{F}{A} ; A = area , F = force \\ Strain ( ɛ ) in \% \text{:} ɛ = \frac{Δ l}{{l}_{0}} ; Δ l = displacement ; {l}_{0} = inital length \\ Young ’ s modulus ( E ) in MPa \text{:} E = \frac{σ}{ɛ}$$

$$Stiffness ( κ ) in N \text{/} mm \text{:} κ = \frac{Δ F}{Δ l}$$

For non-implanted scaffold material preconditioning was performed between 20 N and 500 N. Furthermore, subsequent creep testing until equilibrium was performed at a constant load of 500 N. Data were analysed as described above. Additionally, the equilibrium modulus E_{eq} in MPa was determined:

$${E}_{eq} = \frac{σ}{{ɛ}_{t \to ∞}}$$

### Specimens

After three months of implantation, each tendon was examined macroscopically. After macroscopic examination, the tendons were randomly assigned to different testing groups (Table 3):

TABLE 3
Overview of the different testing groups.

| | |
|---|---|
| Intervention group: Implant (n = 6) | Right (operated) tendon |
| Intervention group: Control (n = 6) | Right (operated) tendon |
| Native Tendon (n = 6) | Left (non-operated) tendon |
| Primary tendon suture (in vitro) (n = 5) | Left (non-operated) tendon |
| Implantation of the scaffold (in vitro) (n = 5) | Left (non-operated) tendon |

### Results

Two animals were excluded from the study due to complications during anaesthesia (control-group) and in the postoperative period (scaffold-group), respectively.

The remaining 16 animals recovered well after surgery. During the first postoperative days, there was a slight swelling and redness of the surgical area. In the further course of the study, animals displayed no abnormalities in movement and there were no macroscopically visible signs of inflammation, i.e. swelling, redness or wound exudation of the operated hind limb.

Scaffold-group: During preparation, two animals of the scaffold-group were excluded, as the scaffolds were dislocated and not detectable around the defect. Although there were no pathological changes of the skin in the surgical area, in one animal of the scaffold-group there were signs of inflammation (i.e. redness) around the tendon graft. In four animals, the scaffold was dislocated proximally to the intraoperative position. However, there were no adhesions to the subcutaneous tissue. A possible reason for the proximal dislocation of some implants might be a rupture of the distal suture and subsequent contraction of the grafted tendon.

Control-group: In the control-group, the operated tendons showed multiple adhesions with the paratendon and the subcutaneous tissue. Moreover, the tendon was broader and had a more flattened appearance compared to the native tendon of the non-operated left leg.

All samples were subjected to a macroscopic scoring system according to Stoll et al. (2011). Thereby a score between 0 and 17 was assigned to each sample, assessing inter alia the overall appearance of the sutured/grafted tendon, adhesion formation to the surrounding tissue and extent of inflammation.

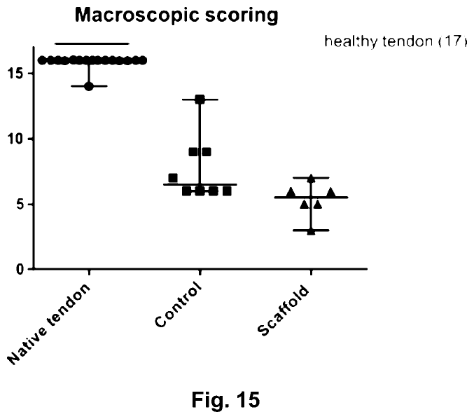

The general appearance of the tendinous tissue was similar in the control and scaffold group. However, there were considerable differences compared to native tendon tissue, especially regarding shape and colour of the tendinous regenerate as well as regarding tendon surface and adhesion formation (FIG. 15).

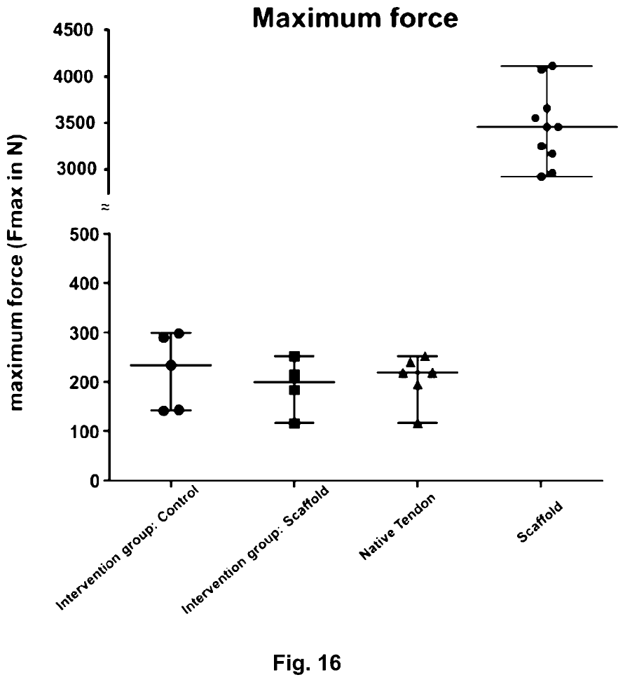

The strength of the native tendon tissue was restored by a tendon suture as well as by tendon grafting with a scaffold. Consequently, the maximum force of native tendon tissue was similar to the maximum force of the control- and scaffold-group (FIG. 16). However, as the maximum force of the non-implanted scaffold material was more than ten times higher, the scaffolds do not seem to contribute to the strength of the healed tendon.

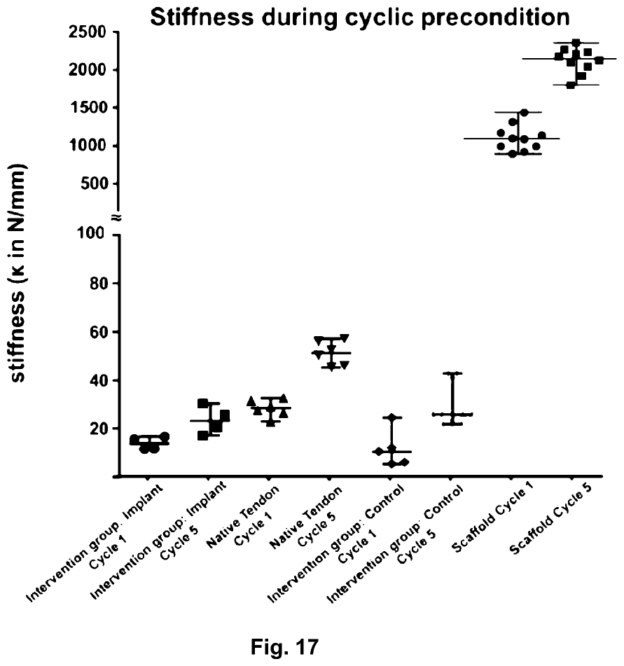

For all specimens, stiffness increased during cyclic preconditioning, suggesting viscoelastic properties (FIG. 17). However, the increase in stiffness over the first five load cycles was less marked in the scaffold- and control-group compared to the native tendon. The non-implanted scaffold material displayed a 30- to 40-fold higher stiffness compared to native tendon tissue (FIG. 17). This huge difference has potentially influenced cellular infiltration and integration of the scaffold in vivo.

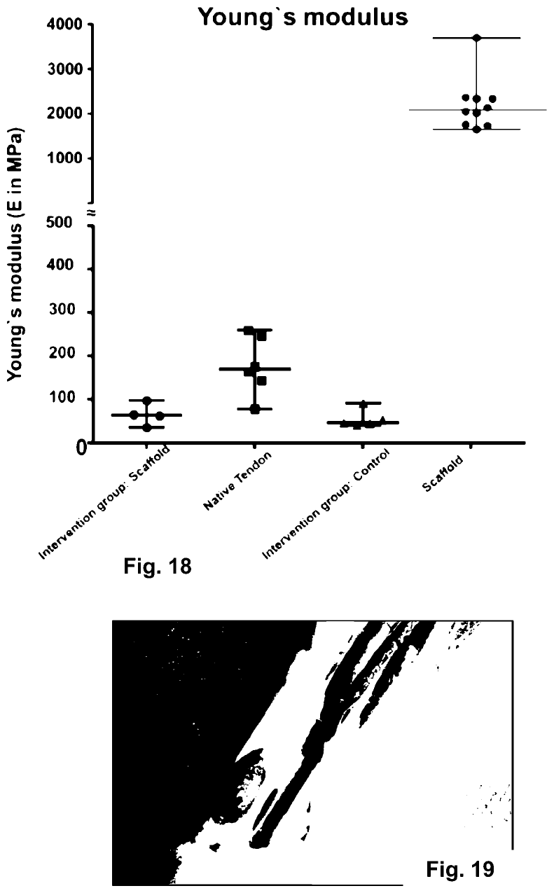

Accordingly, also the Young's modulus of the scaffold material was ten times higher compared to native tissue. Tendinous tissue after suture and scaffold implantation displayed a Young's modulus around 25% of the native tendon tissue (FIG. 18).

In Table 5, maximum stress and maximum strain are summarized for all groups.

TABLE 5
Maximum stress and maximum strain (Median with
minimum and maximum).

| | Maximum stress | Maximum strain |
|---|---|---|
| Group | (σₘₐₓ (in %)) | (εₘₐₓ (in MPa)) |
| Intervention group: | Median: 19.3 | Median: 0.7 |
| Control | Min: 13.9 | Min: 0.3 |
| | Max: 24.4 | Max: 0.8 |
| Intervention group: | Median: 11.2 | Median: 0.3 |
| Implant | Min: 7.9 | Min: 0.3 |
| | Max: 19.0 | Max: 0.4 |
| Native Tendon | Median: 30.1 | Median: 0.20 |
| | Min: 10.7 | Min: 0.2 |
| | Max: 38.3 | Max: 0.3 |
| Scaffold | Median: 175.3 | Median: 0.2 |
| | Min: 132.2 | Min: 0.1 |
| | Max: 215.7 | Max: 0.3 |
| Primary Tendon | Median: 2.3 | Median: 0.4 |
| suture (in vitro) | Min: 1.8 | Min: 0.3 |
| | Max: 2.6 | Max: 0.5 |
| Implantation of the | Median: 1.6 | Median: 0.6 |
| scaffold (in vitro) | Min: 1.2 | Min: 0.50 |
| | Max: 2.0 | Max: 0.9 |

Integration of the scaffold into surrounding native tendon tissue is shown in FIG. 19.

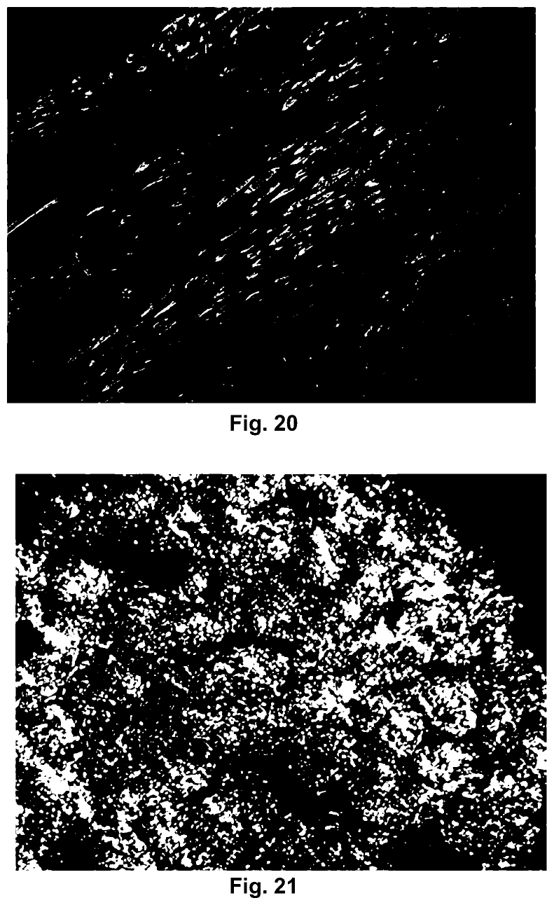

Some exemplary images of non-implanted scaffolds under polarized light are shown in FIG. 20 (longitudinal section of an unstained scaffold, showing fibre arrangement) and FIG. 21 (cross-section of an unstained scaffold, showing fibre arrangement).

### SUMMARY OF EXAMPLES

The examples demonstrate a scaffold comprising PVA hydrogel-coated UHMWPE fibres that can be used as a mechanically strong, purely synthetic scaffold that can be made readily available as off-the shelf products. In particular, the inventors have developed PSG-UHMWPE scaffolds which showed simultaneous high mechanical strength, similar toe-linear modulus, high equilibrium water content, and enhanced oMSC proliferation properties.

The in vivo study results demonstrate the viability of a scaffold of the invention as a tendon implant. In particular, it appears from the in vivo results that the strength of the severed tendons with scaffold implanted is equivalent to both undamaged native tendon, and severed tendon that has been subsequently sutured, likely indicating suitable tissue ingrowth into the scaffold. Also, the in vivo test report notes that there was absence of fibrous tissue adhesion in the tendons with PSG-UHMWPE scaffolds, whereas tendons that had been sutured showed surrounding fibrous tissue adhesion with the underlying subcutaneous tissue layer. This fibrous tissue adhesion is a common clinical issue in tendon repair in humans.

Although the invention has been described with reference to specific examples, it will be appreciated by those skilled in the art that the invention may be embodied in many other forms. In particular, features of any one of the various described examples may be provided in any combination in any of the other described examples.

1. A synthetic implantable scaffold comprising:
a plurality of polymer fibres in contact with a composition comprising:
a hydrogel-forming polymer, and
a biocompatible ceramic material.

2. A scaffold according to claim 1 wherein the synthetic implantable scaffold comprises tensile strength in the range 50 to 170 MPa and/or a tensile modulus in the range of 500 to 2500 MPa.

3. A scaffold according to claim 1, wherein the fibre volume fraction of the scaffold is between about 5-95%.

4. A scaffold according to claim 1, wherein the composition constitutes between about 20-50 wt. % of the synthetic implantable scaffold.

5. A scaffold according to claim 1, wherein the porosity of the scaffold is about 20 to 50 vol. %.

6. A scaffold according to claim 1, wherein the plurality of polymer fibres comprises from 2 to 1000 individual fibres, and wherein the diameter of the individual polymer fibres is between about 1 to about 50 micrometers.

7. A scaffold according to claim 1, wherein the polymer fibres are formed from ultra-high molecular weight polyethylene (UHMWPE).

8. A scaffold according to claim 1, wherein the plurality of individual polymer fibres is in the form of a bundle of fibres having a cross-sectional diameter of between about 150 to 1000 micrometers.

9. A scaffold according to claim 8 further comprising a plurality of bundles of individual polymer fibres having a diameter of between about 1 to 10 mm.

10. A scaffold according to claim 1, wherein at least some of the plurality of polymer fibres are wound or twisted around other fibres to form a yarn or a braid.

11. A scaffold according to claim 1, wherein the implantable scaffold is in the form of a synthetic ligament, wherein the synthetic ligament is selected from the group consisting of: anterior-cruciate ligament, medial collateral ligament, lateral collateral ligament, posterior cruciate ligament, cricothyroid ligament, periodontal ligament, anterior sacroiliac ligament, posterior sacroiliac ligament, sacrotuberous ligament, inferior pubic ligament, superior pubic ligament, suspensory ligament of the penis, suspensory ligament of the breast, volar radiocarpal ligament, dorsal radiocarpal ligament, ulnar collateral ligament, and radial collateral ligament.

12. A scaffold according to claim 1, wherein the implantable scaffold is in the form of a synthetic tendon, wherein the synthetic tendon is selected from the group consisting of: rotator cuff tendon, elbow tendon, wrist tendon, hamstring tendon, patellar tendon, ankle tendon, and foot tendon.

13. A scaffold according to claim 1, wherein the hydrogel-forming polymer is polyvinyl alcohol (PVA), wherein the molecular weight of the PVA is between about 80,000 and about 100,000 g/mol.

14. A scaffold according to claim 1, wherein the hydrogel-forming polymer is present in the composition at between about 5 wt % and about 25 wt %.

15. A scaffold according to claim 1, wherein the composition further comprises a cell adhesion promoter, wherein the cell adhesion promoter comprises gelatin.

16. A scaffold according to claim 15 wherein the concentration of gelatin in the composition is between about 0.1 wt % and about 10 wt %.

17. A scaffold according to claim 16, wherein the ratio of hydrogel-forming polymer:gelatin is between 1:1 to 50:1 (weight %).

18. A scaffold according to claim 1, wherein the biocompatible ceramic material is Hardystonite (Ca₂ZnSi₂O₇) doped with Sr, Mg or Ba, preferably strontium-doped Ca₂ZnSi₂O₇.

19. A scaffold according to claim 18 wherein the strontium-doped Hardystonite is present in the form of microparticles dispersed within the composition.

20. A scaffold according to claim 1, wherein the ratio of hydrogel-forming polymer:biocompatible ceramic material is between 0.5:1 to 10:1.

21. A scaffold according to claim 1, wherein the synthetic implantable scaffold has an equilibrium water content of between about 20 to about 80 wt %.

22. A method for preparing a synthetic implantable scaffold, the method comprising the steps of:
providing a plurality of polymer fibres;
providing a composition comprising: a hydrogel-forming polymer, and a biocompatible ceramic material; and
contacting the plurality of polymer fibres with the composition to thereby form said synthetic implantable scaffold.

23. A method according to claim 22 further comprising the step of providing from 2 to 1000 individual polymer fibres in the form of a bundle of fibres, wherein the bundle of polymer fibres comprises a cross-sectional diameter between about 150 to 1000 micrometers, optionally further comprising the step of winding or twisting at least some of the plurality of polymer fibres around other fibres to form a yarn or a braid.

24. A method according to claim 22, wherein the implantable scaffold is in the form of a synthetic ligament, or in the form of a synthetic tendon.

25. A method according to claim 22, wherein the hydrogel-forming polymer is polyvinyl alcohol having a molecular weight between about 80,000 and about 100,000 g/mol.

26. A method according to claim 22, further comprising the step of providing a cell adhesion promoter comprising gelatine at a concentration between about 0.1 wt % and about 10 wt %.

27. A method according to claim 22, wherein the biocompatible ceramic material is Hardystonite (Ca₂ZnSi₂O₇) doped with Sr, Mg or Ba, preferably strontium-doped Ca₂ZnSi₂O₇.

28. A synthetic implantable scaffold prepared by the method according to claim 22.

29. A method of partial or full tendon or ligament repair in a patient comprising implantation of a synthetic implantable scaffold according to claim 1.

30. Use of a synthetic implantable scaffold according to claim 1 in the manufacture of a medicament for partial or full tendon or ligament repair in a patient.
